(19) United States
(12) Patent Application Publication
(10) Pub. No.: US 2025/0121075 A1
(43) Pub. Date: Apr. 17, 2025

(54) ALPHA-FETOPROTEIN BIOCONJUGATES FOR DISEASE TREATMENT

(71) Applicant: Alpha Cancer Technologies Inc., Toronto (CA)

(72) Inventors: Igor Sherman, Toronto (CA); Mark Frigerio, Cambridgeshire (GB); Anthony Godwin, Cambridgeshire (GB); Jieyu Zhou, Bristol, PA (US)

(73) Assignee: Alpha Cancer Technologies Inc., Toronto (CA)

(21) Appl. No.: 18/291,359

(22) PCT Filed: Jul. 23, 2022
(86) PCT No.: PCT/CA2022/051144

§ 371 Date: Jan. 23, 2024

Related U.S. Application Data
(60) Provisional application No. 63/203,467, filed on Jul. 23, 2021.

(51) Int. Cl. A61K 47/64; A61K 40/11; A61K 40/31; A61K 45/06; A61P 35/00

(52) U.S. Cl. CPC A61K 47/64; A61K 40/11; A61K 40/31; A61K 45/06; A61P 35/00

(57) ABSTRACT

Bioconjugates are described comprising alpha-fetoprotein (AFP) linked with a cytotoxin through a disulfide, glutathione-sensitive linker, for cancer treatment. In a preferred embodiment, the bioconjugate is an AFP variant linked with a maytansinoid cytotoxin via the glutathione-sensitive linker so that the bioconjugate comprises either specific high or low ratio of cytotoxins per AFP.

This application claims priority from U.S. provisional patent application No. 63/203,467, filed Jul. 23, 2021, which is incorporated by reference in this application.

### FIELD OF THE INVENTION

This invention relates to bioconjugates and formulations thereof that are useful in the treatment of cancer and other diseases and conditions. The invention relates particularly to bioconjugates comprising alpha-fetoprotein and a cytotoxin.

### BACKGROUND OF THE INVENTION

Antibody drug conjugates (ADC) are a broad class of medicines in which a cytotoxin is linked to a targeting agent, such as an antibody or other protein (a bioconjugate) that binds selectively to a diseased cell target. The cell binding agent (targeting agent) and the cytotoxin (payload) can be linked covalently via different linker structures. Some linkers provide a cleavage site that is digested intracellularly to release the cytotoxin, once the bioconjugate has entered the cell. Some linkers are inert but are self-immolable and will degrade in stages within the cytosol or lysosomes to release the cytotoxin. The cleavable linkers provide a more controlled and direct drug release mechanism, relying on enzymatic digestion, pH alteration, and other intracellular conditions or agents.

The bioconjugates are composed of a number of highly variable components, and each component requires, for optimal properties, careful selection of each component alone and in combination with the other agent or agents. The targeting agent should bind selectively to the target presented by the diseased cell, so that toxicity is limited to the target site. The bioconjugate should include a toxin that is lethal to or at least damaging to the diseased cell, and the linker should permit release of the cytotoxin when the bioconjugate has entered the cell. The linkage formed between the targeting agent and the bioconjugate should also be cleavable and the number of toxins so linked per targeting molecule is an important parameter. As well, the order in which the components are coupled, and the method by which they are separated (or not) can be key to commercially useful yields. Thus, the bioconjugate itself should be taken up by the cell, and then processed by cellular conditions and components to provide the cytotoxin in a toxic form. It is understood and accepted in this art that a change in any one or more of the main components or steps in bioconjugate design and production can result in significant change, e.g., reduction in the potency or increase in systemic toxicity and other properties of the bioconjugate.

The alpha-fetoprotein (AFP) receptor (AFPR) is highly expressed on tumors and generally displays low or absent expression in healthy tissues with exception of myeloid derived suppressor cells (MDSC), and certain activated lymphocytes which also express the AFP receptors. AFP receptors are present on all embryonic cells, and normally disappear shortly after birth, but are then re-expressed in most adult and pediatric cancers. In the fetus, AFP functions as a shuttle protein, similar to albumin in adults, bringing amino acids, fatty acids and other needed molecules into the embryonic cells by reversibly binding to these molecules and then delivering them into the cell via the AFP receptor. Once inside the cell, AFP releases nutrients into the cell and then returns into circulation to resume shuttling additional molecules into the cell.

AFP bioconjugates in the prior art include a preparation in which AFP is complexed non-covalently with a taxane as an anti-cancer agent (see WO2016/119045 published 4th August 2016). No linker is used, and the complex is expected to penetrate the cell and then release the taxane to effect treatment. There is also mention of AFP-based bioconjugates that incorporate a drug maytansine (DM), known also as a maytansinoid, linked using a non-cleavable linker or bridge. The maytansinoids are themselves cytotoxins that are structurally similar to rifamycin, geldanamycin and ansatrienin. Maytansinoids can bind tubulin and interfere with the formation of microtubules inducing mitotic arrest in the “intoxicated” cells.

Other disclosures relate to AFP bioconjugates and toxic payloads that use a conjugating linker, but one that is non-cleavable; see Merrimack U.S. Pat. No. 7,208,576 published Apr. 24, 2007. In this conjugate, the tacatuzumab AFP antibody, and the DM-1 are coupled between a non-cleavable linker i.e., N-succinimidyl-4-(N-maleimidomethyl) cyclohexane-1-carboxylate (SMCC). The DM-1 toxin is (N2′-Deacetyl-N2′-(3-mercapto-1-oxopropyl) maytansine. Other toxins and linkers useful with AFP include those described by Polytherics in WO2018/051109, incorporated herein by reference, where a disulfide linker that is glutathione-cleavable is coupled with a new variety of toxic agents, including cytotoxins that are DM-like but comprise a phenyl group that substitutes for a chloro group. A conjugate in which AFP is coupled with daunorubicin is also described by Belyaev et al in Cancer Immunol Immunother., 2017. This work is of particular interest in demonstrating a positive effect for AFP-based bioconjugates against myeloid-derived suppressor cells (MDSCs), a subset of immune cells that have been shown to express AFPR. In this study, treatment of mice bearing Ehrlich carcinoma with AFP-doxorubicin resulted in reduced numbers of splenic MDSCs, normalization of NK cell levels, and inhibition of tumor growth. The obtained results demonstrate that cytotoxic bioconjugates based on AFP are promising anti-MDSC drugs, in another embodiment, in addition to their direct effect on tumor cells.

### SUMMARY OF THE INVENTION

It would be desirable to provide a composition for delivering a toxin to diseased cells that present the AFP receptor (AFPR) on their surface which has minimal or no toxicity to normal cells. It would also be desirable to provide a treatment and pharmaceutical composition for treating cells that are selectively responsive to a cytotoxin that can be released in the cytosol by cleavage with glutathione.

There are now provided bioconjugates useful in the treatment of AFPR positive (AFPR+) diseased cells, including AFPR+ cancer cells such as hematopoietic cancer cells and solid tumor cancer cells, including cancer stem cells. Non-cancer cells that are AFPR+, such as myeloid-derived suppressor cells, also can be treated with the bioconjugates disclosed in this application. In these bioconjugates, an AFPR binding agent, such as an AFP, is conjugated to a cytotoxin using a glutathione-cleavable i.e., glutathione-sensitive, disulfide linker. With careful selection of component species, including payload/toxin, linker and AFPR targeting agent, these bioconjugates perform extremely well in efficiently and selectively releasing the glutathione-cleaved cytotoxin in the cytosol and in toxic form and concentration, as desired. The presently preferred bioconjugates show a significant improvement in many parameters, such as in reducing or inhibiting the growth of colorectal tumor xenografts in the COLO-205 mouse model.

In accordance with the present invention, there is provided a bioconjugate useful in the treatment of diseased cells, comprising:
(1) an agent that binds to alpha-fetoprotein (AFP) receptor (AFPR) on the surface of an AFPR⁺ cell;
(2) a cytotoxin (CT) that is toxic to the AFPR+ cell, and
(3) a glutathione-cleavable disulfiiode linker, wherein the linker
   (i) is coupled covalently between the AFPR-binding agent and the CT, and
   (ii) releases the CT intracellularly to the AFPR+ cell.

In one aspect, the AFPR-binding agent has the amino acid sequence of mature, wildtype AFP, of an AFPR-binding fragment or of AFPR-binding variant of the mature wildtype AFP or of the AFP fragment. In another aspect, the AFP is recombinant human AFP, or an AFPR-binding fragment or variant thereof, the variant comprising from 1-5 amino acid substitutions. In yet another aspect, the agent is an AFP that lacks glycosylation. In still another aspect, the agent is recombinant [Asn233Gln] mature human AFP comprising SEQ ID No.3. In another aspect, the agent comprises SEQ ID No.4. In another aspect, the recombinant [Asn233Gln] human AFP is produced by transgenic bacteria. In still another aspect, the recombinant [Asn233Gln] human AFP is produced by a transgenic mammal including a transgenic goat.

In another aspect of the invention, the cytotoxin (CT) has the formula:

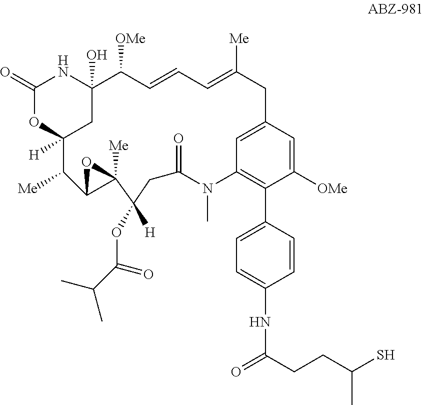 [SMILES: COc1cc2cc(c1-c1ccc(NC(=O)CCC(C)S)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

In another aspect of the invention, the linker coupled between the AFPR-binding agent and the cytotoxin is selected from:

—S—CH(CH3)-(CH2)₂-CO—; and Linker 1

—S—(CH2)₃-CO—. Linker 2

In still another aspect, the conjugate comprises the linker-cytotoxin shown below

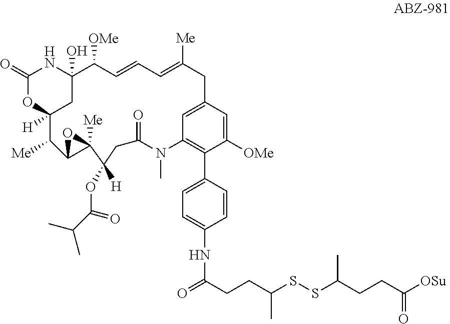

In yet another aspect of the invention, the conjugate comprises the linker-cytotoxin shown below:

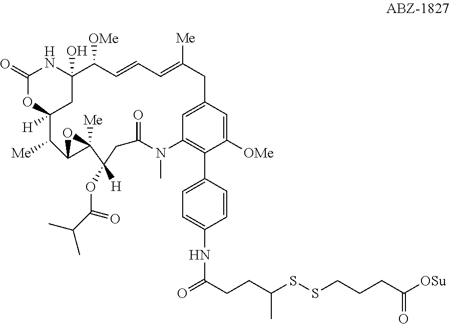

In accordance with the present invention, there is provided a bioconjugate of the formula:

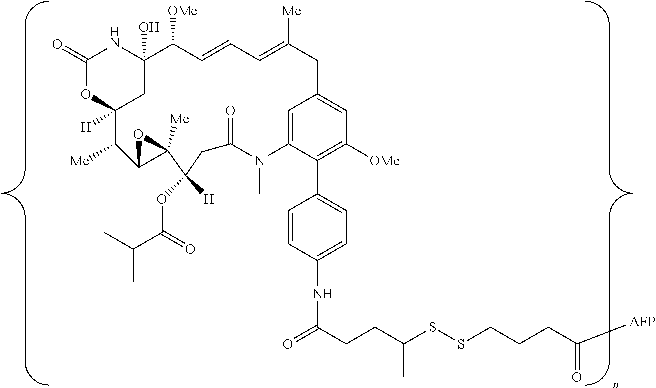

wherein AFP is an AFP receptor-binding form of alpha-fetoprotein.

In an aspect of the invention, n lies in the range from 1 to 11. In another aspect, the average DPR, where DPR is defined as Drug to Protein ratio, is between 2 to 8. In another aspect, the average DPR is between 5 to 7. In yet another aspect, the average DPR is between 5.6 and 6.1. In still another aspect, the average DPR is about 5.9. In yet another aspect, the average DPR is between 3 to 4.5. In another aspect, the average DPR is between 3.6 and 4.1. In still another aspect, the average DPR is about 3.9.

In another aspect of the invention, the AFP is recombinant [Asn233Gln] mature human AFP comprising SEQ ID No.3. In an aspect of the invention, n lies in the range from 1 to 11. In another aspect, the average DPR is between 2 to 8. In another aspect, the average DPR is between 5 to 7. In still another aspect, the average DPR is between 5.6 and 6.1. In yet another aspect of the invention, the average DPR is about 5.9. In still another aspect, the average DPR is between 3 to 4.5. In still another aspect, the average DPR is between 3.6 and 4.1. In yet another aspect, the average DPR is about 3.9.

In accordance with the present invention, there is provided a bioconjugate of the formula:

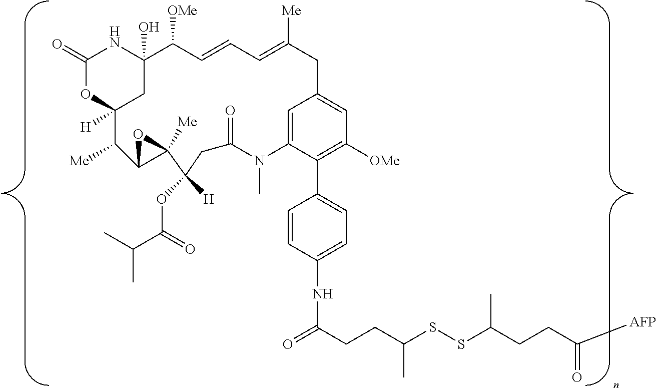

wherein APF is an AFP receptor-binding form of alpha-fetoprotein.

In an aspect of this invention, the n lies in the range from 1 to 11. In another aspect, the average DPR is between 2 to 8. In another aspect, the average DPR is between 5 to 7.

In another aspect, the average DPR is between 5.5 and 6.1. In still another aspect, the average DPR is about 5.8. In yet another aspect, the average DPR is between 3 to 4.5. In another aspect, the average DPR is between 3.4 and 4.0. In still another aspect, the average DPR is about 3.7.

In yet another aspect, there is provided a pharmaceutical composition comprising a pharmaceutically acceptable carrier and a bioconjugate as set out above. In another aspect, the carrier is an aqueous vehicle. In another aspect, the aqueous vehicle is 10 mM HEPES buffer at pH 7.5 with 5% sucrose. In still another aspect, the pharmaceutical composition is for administration into a subject suffering from a cancer that is AFPR+.

In another aspect, the pharmaceutical composition is for administration into a subject suffering from a cancer that is AFPR+.

In another aspect, there is provided a method for treating AFPR+ cells in a subject in need thereof, the method comprising administering to the subject a treatment effective amount of a pharmaceutical composition as set out above. In one aspect, the AFPR+ cells are cancer cells. In another aspect, the AFPR+ cells are MDSCs. In another aspect, the AFPR+ cells are ovarian cancer cells. In still another aspect, the AFPR+ cells are colorectal cancer cells. In still another aspect, the AFPR+ cells are breast cancer cells. In yet another aspect, the AFPR+ cells are lymphoma cells.

In another aspect of the invention, there is provided for use in therapeutic combination, for the treatment of subjects suffering from cancers that are AFPR+, a pharmaceutical composition as set out above and a second treatment agent. In an aspect, the second treatment agent is an immune checkpoint inhibitor or a CAR-T agent or another immuno-oncology therapy.

In another aspect of the invention, there is provided a process for producing a bioconjugate, comprising coupling an AFPR-binding form of AFP as set out above with a conjugate as set out above.

In another aspect of the invention, there is provided a process for producing a bioconjugate whereby the cytotoxin ABZ-981 is first coupled with a glutathione-sensitive disulfide linker to produce an intermediate:

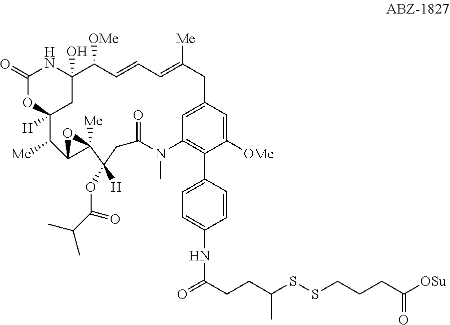

that is then reacted with an AFPR-binding form of AFP that is recombinant [Asn²³³Gln] mature human AFP comprising SEQ ID No.3, and the intermediate is coupled covalently to the epsilon amino groups of lysine residues in the AFP.

In another aspect of the invention, there is provided a process for producing a bioconjugate whereby the cytotoxin ABZ-981 is first coupled with a glutathione-sensitive disulfide linker to produce an intermediate:

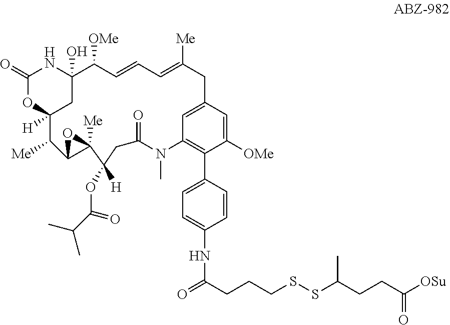

### BRIEF DESCRIPTION OF THE DRAWINGS

These and other aspects of the present invention are now described in greater detail with reference to the accompanying figures in which:

FIG. 1 shows the specific binding of the bioconjugates to the AFP receptor on U-937 cells.

FIG. 2 shows the effect of AFP-cytotoxin bioconjugates on tumor growth in mice bearing COLO-205 colorectal cancer xenografts.

FIG. 3 shows the effect of AFP-cytotoxin bioconjugates on survival in mice bearing COLO-205 colorectal cancer xenografts.

FIG. 4 shows the effect of ACT-903 on tumor growth in mice bearing A2780 ovarian cancer xenografts.

FIG. 5 shows the effect of ACT-903 on survival in mice bearing A2780 ovarian cancer xenografts.

### DETAILED DESCRIPTION

The present invention provides pharmaceutically useful bioconjugates in which a cytotoxic drug (known also as a cytotoxin (CT), payload or a warhead) is coupled covalently to any agent that binds AFPR target on the surface of targeted cells. The targeting agent is an AFPR-binding form of alpha fetoprotein, abbreviated AFP. The linkers providing the covalent coupling between cytotoxin and targeting agent also are sensitive to cleavage by intracellular glutathione, thereby providing a mechanism for the intracellular release of the cytotoxin from the AFP that delivered it there, while remaining stable in blood. Herein, the linkers are described as glutathione-sensitive, which is intended to mean that the linkers can be cleaved by glutathione especially as it exists in the cytosol of a diseased cell to be treated. The linkers are said to be disulfide, because they incorporate an —S—S— arrangement in their structure. These linkers are also stable in plasma with only minimal loss of payload over a period of several days.

The AFP used in the bioconjugates, in its natural state, is a human transporter protein first produced in the fetus by embryonic liver and yolk sac. It enters the cells by endocytosis following binding to the specific AFP receptor. Other forms of AFP, including peptides that constitute an AFPR binding domain, that have these AFPR-binding and transporter properties could be useful targeting agents in the present bioconjugates.

In particular embodiments the present invention provides bioconjugates in which an AFPR-binding alpha-fetoprotein (AFP) in wild type (UniProt KB P02771) or in variant, truncated, fragmented or fragment form, including particularly [Asn233Gln] mature human AFP[1-591], is provided as the AFPR binding agent. In other embodiments this binding agent is linked covalently, through one of two disulfide linkers noted, to a cytotoxin.

The terms “alpha-fetoprotein”, “alfa-fetoprotein”, and “AFP” are used interchangeably herein with reference to the secreted human protein having the 591-mer, mature sequence (residues 19-609) set out in UniProtKB designation P02771. The actual sequence of mature human AFP is shown below as a 609-mer that, in mature form, and lacking the secretion signal (residues 1-18), is reduced to 591 residues in all:

| SEQ ID No. 1 | |
|---|---|
| MKWVESIFLI FLLNFTESRT LHRNEYGIAS ILDSYQCTAE ISLADLATIF | 50 |
| FAQFVQEATY KEVSKMVKDA LTAIEKPTGD EQSSGCLENQ LPAFLEELCH | 100 |
| EKEILEKYGH SDCCSQSEEG RHNCFLAHKK PTPASIPLFQ VPEPVTSCEA | 150 |
| YEEDRETFMN KFIYEIARRH PFLYAPTILL WAARYDKIIP SCCKAENAVE | 200 |
| CFQTKAATVT KELRESSLLN QHACAVMKNF GTRTFQAITV TKLSQKFTKV | 250 |
| NFTEIQKLVL DVAHVHEHCC RGDVLDCLQD GEKIMSYICS QQDTLSNKIT | 300 |
| ECCKLTTLER GQCIIHAEND EKPEGLSPNL NRFLGDRDFN QFSSGEKNIF | 350 |
| LASFVHEYSR RHPQLAVSVI LRVAKGYQEL LEKCFQTENP LECQDKGEEE | 400 |
| LQKYIQESQA LAKRSCGLFQ KLGEYYLQNA FLVAYTKKAP QLTSSELMAI | 450 |
| TRKMAATAAT CCQLSEDKLL ACGEGAADII IGHLCIRHEM TPVNPGVGQC | 500 |
| CTSSYANRRP CFSSLVVDET YVPPAFSDDK FIFHKDLCQA QGVALQTMKQ | 550 |
| EFLINLVKQK PQITEEQLEA VIADFSGLLE KCCQGQEQEV CFAEEGQKLI | 600 |
| SKTRAALGV; secretable wild type human AFP (1-609); | |
| SEQ ID No. 2 | |
| RTLHRNEYGI ASILDSYQCT AEISLADLAT IFFAQFVQEA TYKEVSKMVK | 50 |
| DALTAIEKPT GDEQSSGCLE NQLPAFLEEL CHEKEILEKY GHSDCCSQSE | 100 |
| EGRHNCFLAH KKPTPASIPL FQVPEPVTSC EAYEEDRETF MNKFIYEIAR | 150 |
| RHPFLYAPTI LLWAARYDKI IPSCCKAENA VECFQTKAAT VTKELRESSL | 200 |
| LNQHACAVMK NFGTRTFQAI TVTKLSQKFT KVNFTEIQKL VLDVAHVHEH | 250 |
| CCRGDVLDCL QDGEKIMSYI CSQQDTLSNK ITECCKLTTL ERGQCIIHAE | 300 |
| NDEKPEGLSP NLNRFLGDRD FNQFSSGEKN IFLASFVHEY SRRHPQLAVS | 350 |
| VILRVAKGYQ ELLEKCFQTE NPLECQDKGE EELQKYIQES QALAKRSCGL | 400 |
| FQKLGEYYLQ NAFLVAYTKK APQLTSSELM AITRKMAATA ATCCQLSEDK | 450 |
| LLACGEGAAD IIIGHLCIRH EMTPVNPGVG QCCTSSYANR RPCFSSLVVD | 500 |
| ETYVPPAFSD DKFIFHKDLC QAQGVALQTM KQEFLINLVK QKPQITEEQL | 550 |
| EAVIADESGL LEKCCQGQEQ EVCFAEEGQK LISKTRAALG V; | 591 |
| [wildtype mature human AFP (1-591] | |
| SEQ ID No. 3 | |
| RTLHRNEYGI ASILDSYQCT AEISLADLAT IFFAQFVQEA TYKEVSKMVK | 50 |
| DALTAIEKPT GDEQSSGCLE NQLPAFLEEL CHEKEILEKY GHSDCCSQSE | 100 |
| EGRHNCFLAH KKPTPASIPL FQVPEPVTSC EAYEEDRETF MNKFIYEIAR | 150 |
| RHPFLYAPTI LLWAARYDKI IPSCCKAENA VECFQTKAAT VTKELRESSL | 200 |
| LNQHACAVMK NFGTRTFQAI TVTKLSQKFT KVQFTEIQKL VLDVAHVHEH | 250 |
| CCRGDVLDCL QDGEKIMSYI CSQQDTLSNK ITECCKLTTL ERGQCIIHAE | 300 |
| NDEKPEGLSP NLNRFLGDRD FNQFSSGEKN IFLASFVHEY SRRHPQLAVS | 350 |
| VILRVAKGYQ ELLEKCFQTE NPLECQDKGE EELQKYIQES QALAKRSCGL | 400 |
| FQKLGEYYLQ NAFLVAYTKK APQLISSELM AITRKMAATA ATCCQLSEDK | 450 |
| LLACGEGAAD IIIGHLCIRH EMTPVNPGVG QCCTSSYANR RPCFSSLVVD | 500 |
| ETYVPPAFSD DKFIFHKDLC QAQGVALQTM KQEFLINLVK QKPQITEEQL | 550 |
| EAVIADESGL LEKCCQGQEQ EVCFAEEGQK LISKTRAALG V, | 591 |
| [Asn²³³Gln] rhAFP (1-591) | |
| [SEQ ID No. 4] | |
| MKWVESIFLI FLLNFTESRT LHRNEYGIAS ILDSYQCTAE ISLADLATIF | 50 |
| FAQFVQEATY KEVSKMVKDA LTAIEKPTGD EQSSGCLENQ LPAFLEELCH | 100 |
| EKEILEKYGH SDCCSQSEEG RHNCFLAHKK PTPASIPLFQ VPEPVTSCEA | 150 |
| YEEDRETFMN KFIYEIARRH PFLYAPTILL WAARYDKIIP SCCKAENAVE | 200 |
| CFQTKAATVT KELRESSLLN QHACAVMKNF GTRTFQAITV TKLSQKFTKV | 250 |
| XFTEIQKLVL DVAHVHEHCC RGDVLDCLQD GEKIMSYICS QQDTLSNKIT | 300 |
| ECCKLTTLER GQCIIHAEND EKPEGLSPNL NRFLGDRDFN QFSSGEKNIF | 350 |
| LASFVHEYSR RHPQLAVSVI LRVAKGYQEL LEKCFQTENP LECQDKGEEE | 400 |
| LQKYIQESQA LAKRSCGLFQ KLGEYYLQNA FLVAYTKKAP QLTSSELMAI | 450 |
| TRKMAATAAT CCQLSEDKLL ACGEGAADII IGHLCIRHEM TPVNPGVGQC | 500 |
| CTSSYANRRP CFSSLVVDET YVPPAFSDDK FIFHKDLCQA QGVALQTMKQ | 550 |
| EFLINLVKQK PQITEEQLEA VIADFSGLLE KCCQGQEQEV CFAEEGQKLI | 600 |

SKTRAALGV [609]
X²⁵¹ is Gln, i.e., [Asn²⁵¹Gln] rhAFP (1-609)

It will be appreciated that AFP activity, such as AFPR binding activity and transporter activity, should be retained in AFP variants that incorporate one or more amino acid substitutions, deletions or addition, including particularly conservative amino acid substitutions in the whole protein or fragments that bind AFPR. These can be referred to as mutants or variants of AFP or its AFPR-binding fragments. The alterations can introduce or eliminate post translational modification sites such as glycosylation sites, enzyme vulnerability and the like.

Useful in the present method are those forms of AFP that bind to the AFP receptor, and occur either in a natural state or as natural variants, such as the Lys187Gln variant, and an Asn233Gln variant. As well, the present invention embraces the use of any fragments of AFP that retain AFPR binding, and variants of any such fragments.

Also useful herein are post-translationally modified forms of AFP, including those that incorporateglycosylation. In the human form, N-linked glycosylation occurs at Asn233. Thus, the natural form of human AFP is suitable for use in the present invention. Also useful are non-glycosylated forms of human AFP such as may be produced in prokaryotic host cells such as E. coli and Streptomyces, provided proper 3-D folding enabled by 16 disulfide bridges in native human AFP is maintained. As well, alternatively glycosylated forms of human AFP are useful herein, such as those forms that can be produced in eukaryotic hosts including yeast, Aspergillus, Pichia, insect cells and the like, and in mammalian cell hosts that include CHO and COS cells. Also, a form of human AFP that is especially suitable for the present invention is a human AFP form that is produced in transgenic animals, including goats, rabbits and in some cases pigs. Production of recombinant human AFP in transgenic animals generally, and in the milk of goats specifically, is described for instance in Merrimack's U.S. Pat. No. 7,208,576, which further describes the production of an unglycosylated form of mature AFP that incorporates an Asn233Gln substitution.

In a preferred embodiment, the AFP is a recombinant form of human alpha fetoprotein that is a non-glycosylated mature form of human alpha fetoprotein (hAFP) produced in transgenic goats from a goat expression system. “ACT-101” is a useful species of AFP that differs from naturally occurring human AFP in that it contains one amino acid substitution at amino acid 233 of the mature sequence (glutamine for asparagine). Essentially the same recombinant AFP can be produced, in a non-glycosylated form, by E. coli, where expression is driven from the trp and mal systems, for instance.

The target for AFP binding is the alpha fetoprotein receptor (AFPR). This is typically associated with fetal tissue and is not present on adult cells, more than two months after birth. However, a large proportion of cancer cells express functional AFPR. Although this receptor has only been partially characterized, its existence has been unequivocally supported by experimental evidence. Spontaneously arising tumors in mice or human tumor xenografts implanted in mice have been shown to take up radiolabeled forms of AFP, using a Tc-99m form rhAFP as described in Cancer Biother Radiopharm 1999, 14(6):485-94.

It will thus be appreciated that diseased cells targeted by the present bioconjugates are identified either by their reactivity with AFPR antibodies, or by their binding affinity for AFP itself. These cell targets can be characterized as being AFPR positive, or as having AFP-binding affinity.

Using such reagents, it will also be appreciated that different forms of AFP useful to deliver cytotoxic drugs, to a target diseased cell can be identified by their ability in equimolar terms to displace labeled AFP from binding with AFPR for diagnostic purposes, or by their ability to engage AFPR directly. Cell-based assays are also useful for this purpose. In one example, AFPR-expressing U937 cells (a human male histiocytic lymphoma cell line available from ATCC under catalog number CRL 1593.2TM) are exploited to confirm the AFPR binding affinity of any given form of AFP or AFP bioconjugate.

In one embodiment, there are provided bioconjugates formed by covalently linking AFP and a cytotoxin. Cytotoxins are very well-known, very broad class of agents that are used in a variety of drug conjugates or as single therapeutic agents. In the present bioconjugates, the AFP component of the bioconjugate is coupled or bridged to a cytotoxin that is not DM-1, DM-3 or DM-4, but shares some structural aspects with these cytotoxins. Cytotoxins useful in the present invention include:

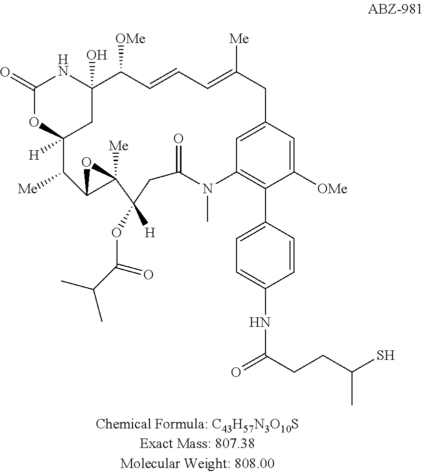 [SMILES: COc1cc2cc(c1-c1ccc(NC(=O)CCC(C)S)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

These maytansinoid cytotoxin payloads in the present bioconjugates are attached to the recombinant human AFP (rhAFP) using a linker containing a cleavable glutathione sensitive disulfide bridge. Glutathione is a thiol-containing coenzyme integrally involved in many thiol-disulfide redox processes. In its reduced (thiol) form, glutathione is abbreviated ‘GSH’. In its oxidized form, glutathione exists as a dimer of two molecules linked by a disulfide group, and is abbreviated ‘GSSG’. Disulfide bonds and free thiol groups in target proteins and glutathione can ‘trade places’ through a disulfide exchange reaction. This process is essentially a combination of two direct displacement events, with sulfur atoms acting as nucleophile, electrophile and leaving group, to cause cleavage of the linker disulfide and release of the toxin. Intracellular glutathione concentrations usually range from 0.5 to 10 mM, whereas extracellular values are substantially lower, about 2 uM in plasma. Additionally, glutathione levels are elevated in tumors, including ovarian cancer so this differential concentration of glutathione can allow for stability of bioconjugates in blood and release of cytotoxin following uptake by tumor cells. Stability of the bioconjugate can be tuned by varying the steric nature of the R groups flanking the disulfide bond. Although other release mechanisms can also be employed (e.g., pH and proteases-sensitive linkages), the glutathione release mechanism is most relevant for rhAFP-toxin bioconjugates, since AFP does not traffic to lysosomes where protease-labile linkers can be cleaved. This has been confirmed by studies on rhAFP-conjugates where bioconjugates with dipeptide, acid-sensitive or non-cleavable linkers showed poor potency in vitro, presumably due to lack of release of the toxin inside the cell, while disulfide-linked maytansine-containing bioconjugates showed relatively high (single digit nM) potency in vitro, which increased proportionally with the number of cytotoxins loaded per molecule of AFP (DPR).

Thus, the AFP and the cytotoxin are bridged, in preferred embodiments, using a linker that forms a glutathione-sensitive disulfide with ABZ-981 having a chemical structure defined as:

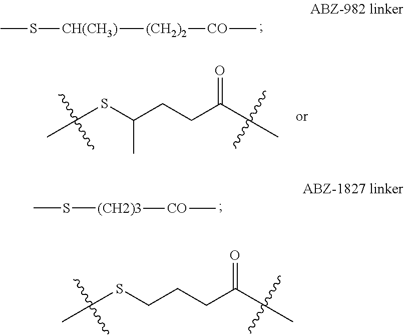

These linkers are formed with a chosen cytotoxin ABZ981 to provide conjugate intermediates that in preferred embodiments have the structure of ABZ-1827 or ABZ-982 as shown below.

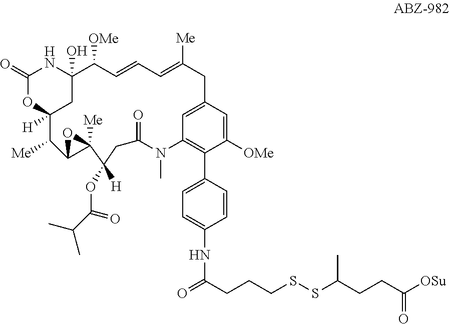

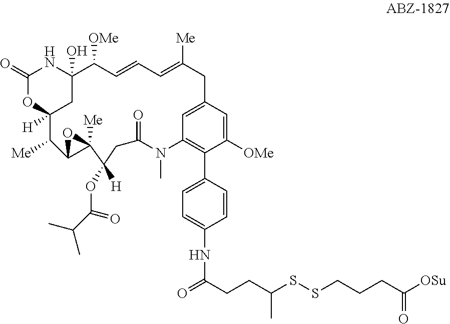

When CT ABZ-981 is coupled through a mono-methylated glutathione-cleavable disulfide linker as taught herein, the resulting linker/payload conjugate, which is useful as a synthetic intermediate, is designated ABZ-1827. Alternatively, when CT ABZ-981 is coupled through a di-methylated glutathione-cleavable disulfide linker as taught herein, the resulting linker/payload conjugate, which is useful as a synthetic intermediate, is designated ABZ-982.

Linker-payloads are synthesized as indicated in the Examples herein. To produce the AFP:cytotoxin bioconjugate, the cytotoxin is first coupled with a glutathione-sensitive disulfide linker to produce an intermediate, for example the intermediates as shown above, that is then reacted with AFP, so that the linker is coupled covalently, usually to the epsilon amino groups of lysine residues in the AFP and, at the other end, to the desired cytotoxin. The isolated bioconjugate can then be mixed within an aqueous vehicle, desirably one that is isotonic, and has a pH that is physiological or mildly more acidic. In one embodiment, the aqueous vehicle is phosphate buffered saline at a pH in the range from about 6 to about 7.5. In another embodiment, the aqueous vehicle is water. In a further embodiment the aqueous vehicle is saline (0.154M NaCl). In a further embodiment the aqueous vehicle is HEPES ((4-(2-hydroxyethyl)-1-piperazineethanesulfonic acid) buffer.

Room temperature and standard pressure are acceptable mixing conditions. The mixing can be fostered using mild agitation, stirring, and the like. In the process, conditions are managed so that a desired average ratio of cytotoxin:AFP is obtained. The ratio is referenced herein as the “average DPR”, i.e., the average Drug:Protein Ratio. In the case of antibody drug conjugates, this would be known as the “Drug Antibody Ratio, DAR”. An average DPR is achieved by controlling the amount of cytotoxin available for reaction with the AFP.

To produce a composition from which calculated unit dosages of cytotoxin can be prepared, the production of the bioconjugate desirably involves the use of predetermined amounts of each reagent. Experiments using Liquid Chromatography-Mass Spectrometry (LC/MS) have found that one molecule of AFP (Molecular weight=66.5 kD) is able to bind and retain about 1-11 molecules of cytotoxin conjugate (Molecular weight=854 kD), when mixing occurs under the conditions noted above. Higher DPR ratios increase the risk of bioconjugate precipitation due to reduced solubility. Thus, an upper limit of DPR is determined by need to maintain bioconjugate in solution without precipitation during storage. Different DPR values can be reached by altering conditions such as the load of each component in terms of molarity or by altering pH of the reaction medium.

It has been determined that the disulfide linkage between the toxin and the protein desirably incorporates a distribution of methyl groups as incorporated in the exemplified and preferred bioconjugates, where the disulfide linkage is either mono-methylated as shown below in ABZ1827-AFP or is di-methylated as shown below in ABZ982-AFP. ABZ1827-AFP is preferred to ABZ982-AFP. This methylation may shield the disulfide linkage, and provides desirable and enhanced balance between bioactivity and stability of the bioconjugates.

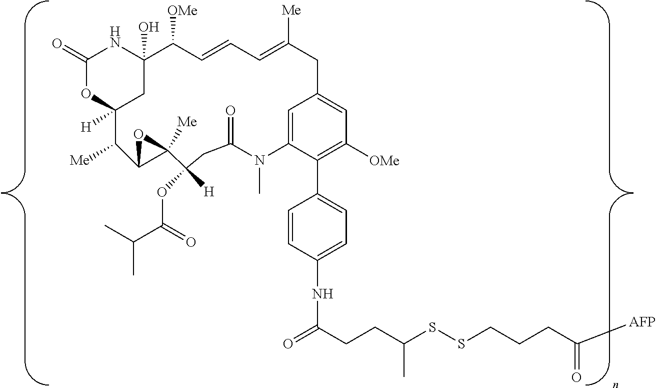

This bioconjugate is referred to herein as ABZ1827-AFP. “n” represents the number of cytotoxin/linker molecules (conjugate intermediates) bound to each molecule of AFP. ABZ1827-AFP will usually contain a range of values of n. The values of n typically range from 1-11; although there may be trace amounts of higher n species.

In another embodiment, the AFP protein is linked covalently to a linker cytotoxin that is shown below:

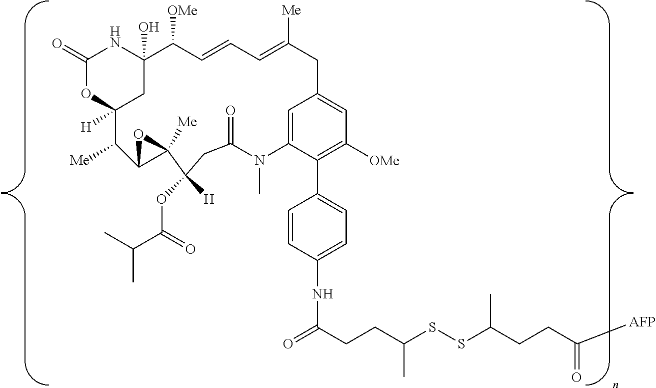

This bioconjugate is referred to herein as ABZ982-AFP. “n” represents the number of cytotoxin/linker molecules (conjugate intermediates) bound to each molecule of AFP. ABZ982-AFP will usually contain a range of values of n. The values of n typically range from 1-11; although there may be trace amounts of higher n species.

The bioconjugates discussed in this application are generally composed of a mixture of bioconjugates with varying n's. The bioconjugates can be prepared that have a more limited range of n, or in other cases have a distribution of n focused around a particular number. Bioconjugates can also be prepared and characterized by an average value of n (also identified as DPR, as defined above).

Thus, the present invention provides in certain embodiments bioconjugates that are the result of coupling the chosen AFP with a cytotoxin/linker that is preferably ABZ982 or ABZ1827, as hereinafter described. The cytotoxin component is coupled to the AFP via a primary amine, such as the epsilon amino group on AFP lysine residues. One, two or more, but not usually more than 11 cytotoxins can be coupled to each AFP molecule in this manner. The ratio of cytotoxins coupled to each AFP protein can also be manipulated to escalate or reduce bioconjugate bioactivity and solubility, depending on this ratio.

The DPR is known to have a bearing on the potency, solubility and the toxicity of bioconjugates. Low DPR values associate with comparably low potency relative to high DPR values for the same bioconjugate. High DPR values sometimes associate with comparably higher toxicity relative to lower DPR values for the same bioconjugate, but not always. The preparation of bioconjugates with different DPR values is achieved by controlling the concentration of cytotoxin provided for coupling to the AFP and duration of the reaction. The greater the amount of cytotoxin relative to AFP, the higher the DPR value that results from the synthesis, with a maximum value reached at a DPR of about 11, and usually less than 11.

In other particular embodiments, the bioconjugates have a DPR that is on average between about 2 and 8 cytotoxin molecules per AFP molecule.

In one specific embodiment, when the cytotoxin/linker is ABZ-982, the DPR is suitably “low”, in the sense that the DPR is on average between 3 and 4.5, or in a more specific embodiment about 3.7+/−0.3.

In another embodiment, the cytotoxin/linker is ABZ-982, the DPR is suitably “high”, in the sense that the DPR is on average between 5 and 7, or in a more specific embodiment about 5.8+/−0.3.

In a further embodiment, when the toxin/linker is ABZ1827, the DPR is suitably “high”, in the sense that the DPR is on average about 5.9+/−0.3 (about 6). In another embodiment, the DPR lies in the range from 5-7 DM per AFP.

In another embodiment, when the toxin/linker is ABZ1827, the bioconjugate has a “low” DPR that is below about 4.5. In a specific embodiment, the DPR is on average about 3.9+/−0.3 (about 4).

As shown in Table 4, ABZ1827-AFP and ABZ982-AFP are more potent in vitro when the DPR is “high” but retains potency when the DPR is “low” as well.

The bioconjugates are useful therapeutically to treat subjects presenting with diseased cells that are AFP-binding, or AFPR-positive. Target cells should also be “cytotoxin-responsive” meaning simply that cells responsive to intracellular cytotoxin show reduced or absent vitality and are either killed, depleted or reduced at least in terms of their number, size, distribution, etc. by the cytotoxin released from the bioconjugate. It has been found that AFP and cytotoxin bind with an affinity that is sufficient, during the course of bioconjugate preparation and following endogenous administration, that the maytansinoid is delivered selectively, and with reduced systemic toxicity, by the associated AFP to the diseased cell. This approach to maytansinoid delivery could allow for reduced dosing of the cytotoxin and consequent reduction in associated adverse events, not only because the bound cytotoxin is less toxic but also because the cytotoxin is delivered selectively to AFPR positive diseased cells, thus sparing normal healthy cells. Moreover, the AFP:cytotoxin bioconjugate can be formulated in benign and standard pharmaceutical vehicles such as saline or HEPES buffer, thereby avoiding the use of carriers that in themselves create toxicity issues upon delivery to the patient.

The bioconjugates can thereafter be formulated immediately for therapeutic administration, stored briefly in its aqueous vehicle, preferably frozen, or lyophilized for prolonged storage, as exemplified herein. Freeze-thaw cycle does not impact aggregation/dimer formation of bioconjugates. The bioconjugates can be stored in solution at 2-8° C. for at least 3 days without degradation. They also can be stored deep frozen (≤−60° C.), and bioconjugates in solution have been shown to retain activity through a freeze/thaw cycle. Thus, in another embodiment, the present invention provides AFP:cytotoxin bioconjugates, in lyophilized or frozen form or refrigerated at 2-8° C. in HEPES buffer.

For therapeutic use, the present invention provides AFP:cytotoxin bioconjugates as a pharmaceutical composition in which the bioconjugate is formulated with a pharmaceutically acceptable carrier. The formulation is adapted, in one embodiment, for intravenous administration, such as by injection or by infusion. Accordingly, the carrier can be an aqueous vehicle such as water for injection, saline, and the like.

The active ingredients to be used for in vivo administration will be sterile. This is accomplished by filtration through sterile filtration membranes.

Any other carriers, vehicles or excipients used in formulating the AFP-conjugated cytotoxin can be chosen to avoid agents or conditions that will disrupt the desired bioconjugate stability or will alter the binding affinity of AFP for AFPR. Organic solvents can be avoided. Agents that introduce a pH outside physiological range, i.e., less than about pH 6 and more than about pH 8, can also be avoided for this reason. The AFP:cytotoxin bioconjugates may be formulated in water, or normal saline or particularly HEPES buffer. Aqueous buffered saline solutions (including phosphate buffered saline) are preferred as they have a physiologically tolerable pH and are adapted for administration by the preferred routes of injection or infusion. Addition of compounds that prevent aggregation or precipitation, such as sucrose, is desirable. Another preferred carrier/vehicle is 10 mM HEPES buffer pH 7.5 with 5% sucrose.

The AFP:cytotoxin bioconjugates are useful therapeutically. Accordingly, and in one aspect, the present invention provides a method for treating a subject presenting with an AFPR positive, or AFP-binding, diseased cell comprising administering to the subject an AFP:cytotoxin bioconjugate comprising AFP-bound cytotoxin in an amount effective to inhibit the growth and/or proliferation of that diseased cell.

AFP bioconjugates are promising anticancer drugs, which, in addition to the direct effect on tumor cells expressing receptors to AFP, may contribute to reduction (or elimination) of MDSCs. These are AFPR+ cells that can support tumor vitality, and are deleterious when present in tumor microenvironment in the same subject. Reduction, depletion or eradication of these cells is a promising pathway for enhancing treatment effect. AFP as a vector molecule conjugated with a cytotoxic agent, specifically recognizes MDSCs and therefore can be used in a cancer patient to reduce the number of MDSCs.

AFP receptor positive diseased cells may be identified for treatment both in vivo and ex vivo, using assays that employ detectable and selective AFP receptor binding ligands. AFPR positive diseased cells that can be targeted by the present bioconjugates include AFPR positive cancer cells, which include generally all cancer cells that bind AFP with specificity. It is anticipated that an effect may be seen only in those AFPR positive diseased cells that respond to cytotoxin with the desired inhibition of growth or proliferation as reflected in reduced tumor size, or reduced tumor growth rate. Such cells and tumors have the property of being “cytotoxin-responsive”, and are the preferred targets for treatment with the present bioconjugate. In addition, certain cytotoxin-resistant cancer cells in which the resistance to cytotoxin is due to overexpression of membrane pumps that actively remove cytotoxin from the cells could be effectively treated with the present AFP:cytotoxin formulations because bioconjugate, after binding to AFPR, crosses the membrane by the process called endocytoses, in which AFPR-bioconjugate are encapsulated in a vesicle and transported to the interior of the cell, thus avoiding interaction with the membrane pumps.

Any appropriate route of administration can be employed, for example, parenteral, including intravenous, intramuscular, subcutaneous, intracranial, intraorbital, intraventricular, intracapsular, intraspinal, intracisternal, intralesional, intratumoral intraperitoneal administration. Intravenous administration by injection or infusion can be preferred.

For the treatment of subjects presenting with cancer cells that bind AFP, the appropriate dosage of an AFP:cytotoxin bioconjugate will depend on the type of disease to be treated, the severity and course of the disease, previous therapy, and the patient's clinical history and response to the agent. The agent is suitably administered to the patient over a series/course of treatments. Progression of disease can be monitored in accordance with practice standards in cancer therapy. Particularly in the case of cancers, effective treatment of a subject in need thereof can result in a reduction in the number, volume, distribution, vitality and other cell parameters including a reduction in the rate of their growth and/or proliferation or maturation.

For example, depending on the type and severity of the diseased, about 20 g/kg to 200 μg/kg of cytotoxin, present in 0.5 mg/kg to 5 mg/kg of AFP bioconjugate if administered once every 3 weeks, is a candidate dosage for administration to the patient, whether, for example, by one or more separate administrations, or by infusion. For repeated administrations once or twice weekly, or every 3 weeks, or over several weeks or longer, depending on the condition, the treatment is sustained until a desired suppression of diseased symptoms occurs or until progression of the diseased is observed. However, other dosage regimens may be useful. Unit doses based on the weight of AFP-cytotoxin bioconjugate can be in the range, for instance of about 500 ug to 500 mg, such as 1 mg, 5 mg, 10 mg, 25 mg, 50 mg, 100 mg, 150 mg, 200 mg, 250 mg, 300 mg and 400 mg. The formulated bioconjugates can be provided in multidose form, comprising 2, 3, 4, 5 or more unit doses within each container, e.g., vial. The bioconjugated preparations can also be provided in kit form, comprising a lyophilized or frozen preparation comprising the bioconjugate and a separately packaged vehicle for reconstitution of the preparation into an administrable dosage form. In the alternative, the kit may simply comprise the bioconjugated preparation, and instructions for the reconstitution thereof into an administrable dosage form. The progress of anti-cancer therapy is monitored by techniques and assays established for the particular diseased being treated.

For dosing guidance for the present bioconjugates, one should understand that the recommended dose of a marketed bioconjugate, KADCYLA™ (ado-trastuzumab emtansine), is 3.6 mg/kg given as an intravenous infusion every 3 weeks (21-day cycle) until disease progression or unacceptable toxicity, or a total of 14 cycles for patients with breast cancer.

It will thus be appreciated that an effective amount of the bioconjugate is an amount effective as a unit dose or as part of a treatment regimen to retard or inhibit the rate of growth or proliferation of diseased cells and malignancies that are cytotoxin-responsive and positive for AFPR+, including AFPR+ cancer stem cells (CSCs), which are cancer cells (found within tumors or hematological cancers) that possess characteristics associated with normal stem cells, specifically the ability to give rise to all cell types found in a particular cancer sample.

The bioconjugates are useful in the treatment of various cytotoxin-responsive cancers, to inhibit the growth or proliferation of cancer cells and tumors comprising them, including hematological cancers and solid tumors. The specific dose of a composition administered to a subject will depend for example on the administration route, the frequency of administration, the state of the recipient, and the type of cancer being treated. Cancers suitable for treatment are those cancers that express AFP receptor. Demonstrated expression of AFPR has been noted in human cancers or cancer cell lines, including breast, ovarian, colorectal, endometrial, stomach, lung, lymphoma, prostate and liver. Metastases of these cancers can also be treated in accordance with the methods described herein.

The term “subject”, “patient” and “recipient” all refer to mammals including humans in particular but also other primates, livestock, pets, horses and the like. It will be appreciated that the subjects treated with the present bioconjugates should be at least about 3 months old so that endogenous AFP receptor is not prevalent on the subject's healthy cells and tissue. Also, the bioconjugates used to treat non-humans desirably incorporate the form of AFP that is specific for that species.

It is common to administer targeted cytotoxic agents in combination with other therapies, and so a person skilled in the art will appreciate that the bioconjugates can be used in combination with other cancer therapies including immunotherapies.

The bioconjugates can be administered to a subject in need thereof, in combination with useful other therapeutic agents. Administration “in combination with” one or more further therapeutic agents includes simultaneous (concurrent) and consecutive administration in any order. Other therapeutic regimens may be combined with the administration of the anti-cancer agent of the instant invention. For example, the patient to be treated with such anti-cancer agents may also receive radiation therapy, such as external beam radiation. Alternatively, or in addition, a chemotherapeutic or biologic agent may be administered to the patient. Preparation and dosing schedules for such chemotherapeutic or biologic agents may be used according to manufacturers' instructions or as determined empirically by the skilled practitioner. The chemotherapeutic agent may precede, or follow administration or the bioconjugate, or may be given simultaneously therewith. In particular embodiments, bioconjugates can be used in combination with immune-stimulating agents, such as checkpoint inhibitors or CAR-T preparations. since efficacy of such immune-oncology drugs is decreased in the presence of MDSCs. Reduction (or elimination) of MDSCs by AFP bioconjugates should greatly enhance the efficacy of the immuno oncology agents.

The bioconjugates and pharmaceutical compositions of the present invention may if desired be used in combination with an additional therapeutic agent, for example an additional anti-cancer agent, for example, CAR-T agents, immune checkpoint inhibitors, alkylating agents, alkyl sulfonates, aziridines, ethylenimines and methylamelamines, acetogenins, an auristatin, camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antibiotic chromophores, aclacinomysins, actinomycin, authramycin, azaserine, azacitidine, bleomycin, cactinomycin, carabicin, carminomycin, carzinophilin, chromomycinis, dactinomycin, daunorubicin, detorubicin, 6-diazo-5-oxo-L-norleucine, doxorubicin, epirubicin, esorubicin, idarubicin, marcel lomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatrexate, defofamine, demecolcine, diaziquone, eflornithine, elliptinium acetate, an epothilone, etoglucid, gallium nitrate, hydroxyurea, lentinan, lonidainine, maytansinoids, mitoguazone, mitoxantrone, mopidamol, nitraerine, pentostatin, phenamet, pirarubicin, losoxantrone, podophyllinic acid 2-ethylhydrazide, procarbazine, PS® polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chlorambucil; gemcitabine; 6-thioguanine; mercaptopurine; methotrexate; platinum analogs, vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (Camptosar, CPT-1 1), topoisomerase inhibitor RFS 2000; difluorometlhylornithine; retinoids; capecitabine; combretastatin; leucovorin; oxaliplatin; inhibitors (such as antibodies) of PKC-alpha, Raf, H-Ras, EGFR and VEGF-A that reduce cell proliferation; and pharmaceutically acceptable salts, acids or derivatives thereof; or a combination thereof.

The bioconjugates and pharmaceutical compositions of the present invention may also be used in combination with an anti-cancer antibody or polypeptide, for example, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, nanatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, ramucirumab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8 or any combination thereof.

In another embodiment of the invention, an article of manufacture containing AFP:cytotoxin bioconjugate(s) useful for the treatment of the disorders described herein is provided. The article of manufacture comprises the present bioconjugates, in solution or in lyophilized or frozen form, in a container and suitably bearing a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with, the container indicates that the composition is used for treating a cancer condition. The article of manufacture may further compromise a second container comprising a pharmaceutically acceptable buffer, including saline, HEPES-buffer, phosphate-buffered saline, water for injection, and the like. It may further include other matters desirable from a commercial and use standpoint, including other buffers, diluents, filters, needles, syringes, and package inserts with instructions for use in accordance with the present invention. Control agents or standards and calibrators useful in the method can also be included in the kit, such as an AFP preparation standard.

It will thus be appreciated that the alpha fetoprotein (AFP) receptor is an oncofetal antigen and a novel target for cancer therapeutics. This receptor is highly expressed on the surface of many common cancers but is generally not expressed on normal adult cells. By covalently conjugating a novel maytansinoid toxin to a recombinant human form of AFP the toxin payload can be selectively delivered to cancer cells while sparing normal cells. Since AFP is a human protein, there is reduced risk of any harmful immune reaction to AFP bioconjugates.

### Examples of Synthesis

Two forms of the bioconjugate were prepared, differing only in their linker structure (monomethyl vs. dimethyl), and compared in some cases with bioconjugates based on the maytansinoids DM1 and/or DM3 and/or DM4. The general approach to their synthesis is illustrated below, where R is the reagent cytotoxin and P is the protein, AFP.

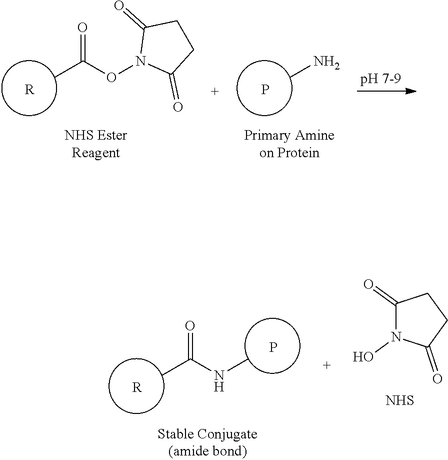

### 1) Preparation of AFP

Mature [Asn²³³Gln]rhAFP(1-591), lacking glycosylation, was produced in transgenic goats and recovered from the goat milk, in the manner described by Merrimack in U.S. Pat. No. 7,208,576 published Apr. 24, 2007 and incorporated herein by reference in full. This form of AFP was designated ACT-101 and has SEQ ID No.3, which incorporates the Asn²³³Gln substitution in mature AFP.

### 2) Preparation of Cytotoxin: Linker Bioconjugate

A maytansinoid-like cytotoxin designated ABZ981 was first produced in the manner described in WO2018/051109, and then covalently coupled with a disulfide glutathione-sensitive linker in the following manner:

General analytical method: Linker-payloads were synthesized as indicated below. All the solvents used were used as is and purchased either from Sigma Aldrich or Fisher Scientific. 1H-NMR spectra were recorded on Varian Inova 500 MHz NMR instruments. The chemical shifts (δ) were reported in ppm with respect to the NMR solvents used for analysis. Coupling constants (J) were reported in hertz (Hz). Chromatographic purities were determined on a Waters UPLC/MS-5SQD system using Kinetex® 1.7 μm C18 100 Å column (50×2.1 mm) and the following analytical UPLC method: injection volume 2-5 μL; flow rate 0.6 mL/min; 10-90% acetonitrile in water over 2.5 to 5 min; ACQUITY UPLC Photodiode Array (PDA) Detector at =254 nm; room temperature. Chromatographic purities were determined on an Agilent 6130, 1260 Infinity, LC/MS systems using Chromolith® FastGradient RP-18e analytical columns (50×2 mm, Merck KGaA, P/N 1.52007.0001)

### Synthesis of Target Compounds

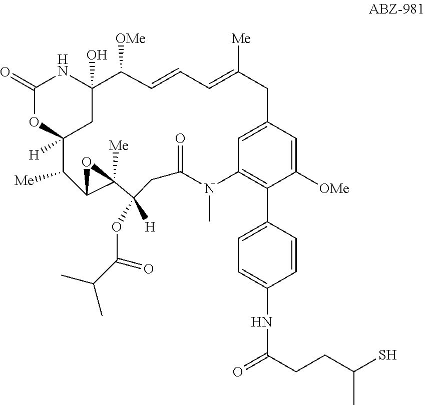 [SMILES: COc1cc2cc(c1-c1ccc(NC(=O)CCC(C)S)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

### Synthetic Scheme:

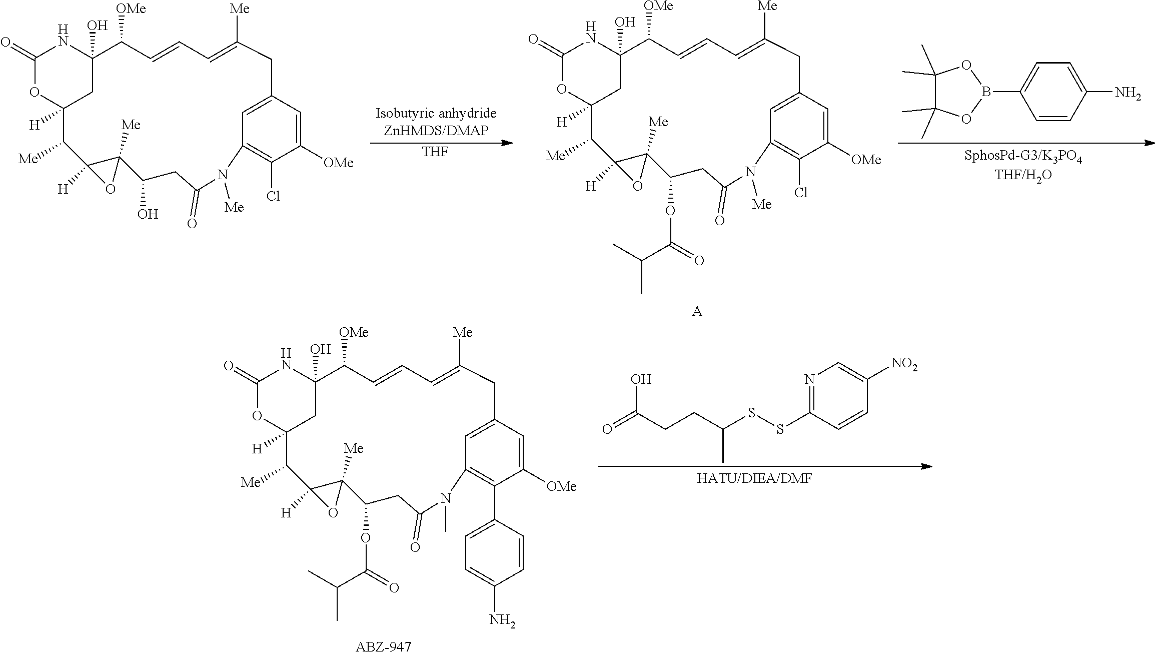

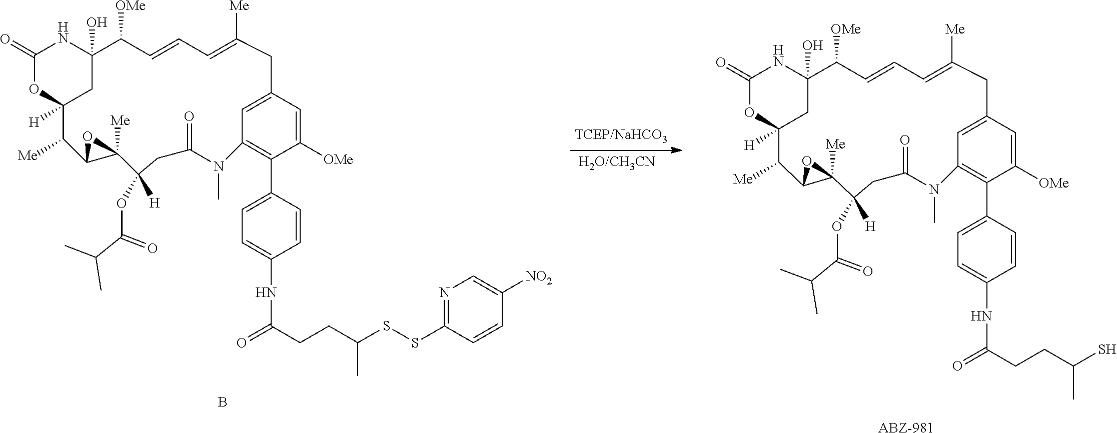

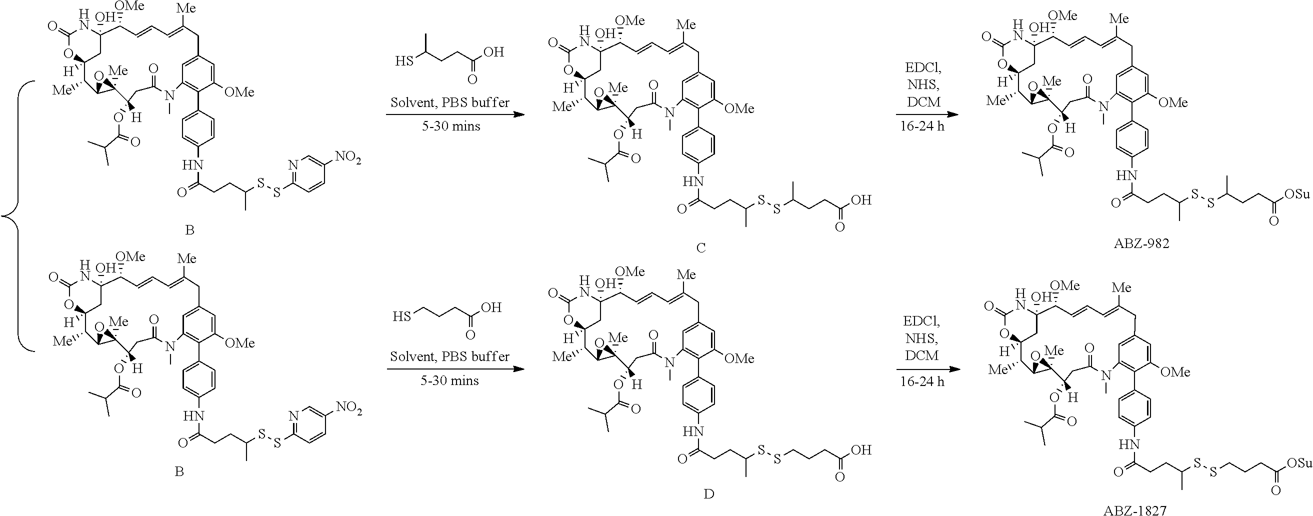

### Experimental Procedures

### Synthesis of Compound a and ABZ-947

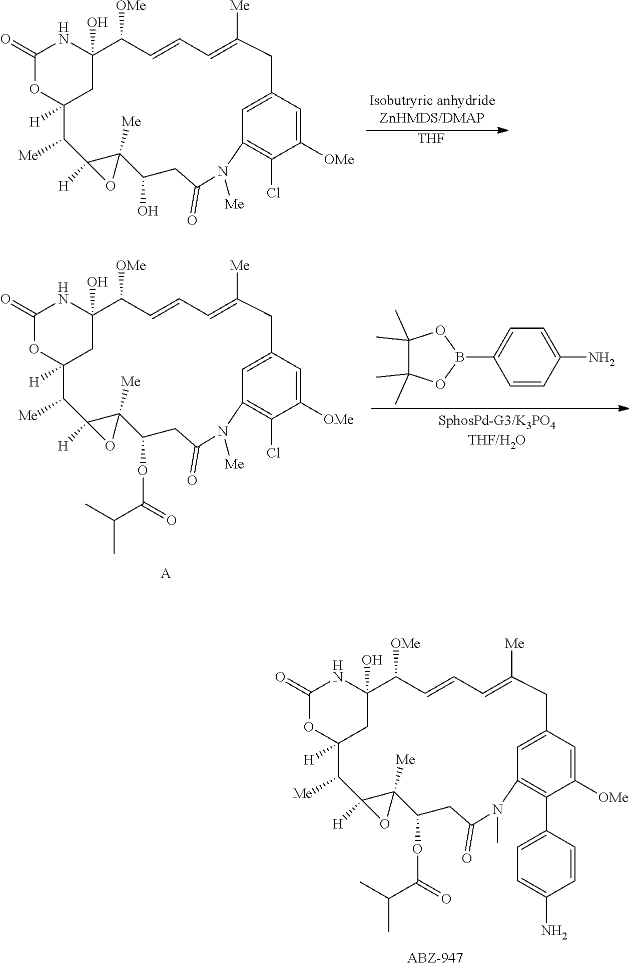

### Compound A:

To a mixture solution of maytansinol (2.6 g, 4.60 mmol) and DMAP (0.56 g, 4.60 mmol) in 26 mL of anhydrous THE at room temperature under argon was added ZnHMDS (4.6 mL, 11.50 mmol) dropwise. The mixture was stirred for 30 min to afford brown suspension solution. To this solution, isobutyric anhydride was added dropwise. The resulting solution was stirred at room temperature for 1.5 h. An aliquot by LC/MS indicated the reaction was complete to give the desired product. The crude was quenched by saturated NH₄Cl solution (100 mL), diluted with 100 mL of ethyl acetate and the organic layer was separated. The aqueous layer was extracted by ethyl acetate (120 mL×2). The combined organics were washed with water (50 mL), brine solution (50 mL), and dried over Na₂SO₄. After concentration, it gave crude brown solid, which was purified by 5% MeOH in DCM using HP 120 g Gold column ISCO system to afford 1.48 g (51%) of comp A. MS (ESI, pos.): Calculated for C₃₂H₄₃ClN₂O₉, 634.27; found 635.3 (M+H), 1293.4 (2M+Na). Another batch was completed and combined for the next step.

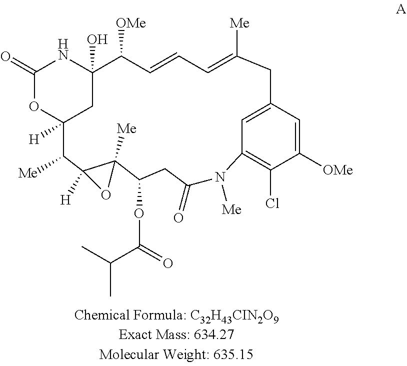 [SMILES: COc1cc2cc(c1Cl)N(C)C(=O)C[C@H](OC(=O)C(C)C)[C@@]1(C)O[C@H]1[C@H](C)[C@@H]1C[C@@](O)(NC(=O)O1)[C@H](OC)/C=C/C=C(\C)C2]

### ABZ-947:

The mixture of water and THF was fully degassed by argon prior to use. To a mixture of compound A (3.10 g, 4.88 mmol), 4-(4,4,5,5-tetramethyl-1,3,2-dioxaborolan-2-yl)aniline (1.60 g, 7.32 mmol), and K₃PO₄ (4.14 g, 19.52 mmol) in 100 mL of round-bottom was charged with THF (50 mL) and H₂O (7 mL). The stirring mixture was degassed by vacuum and refilled with argon. This was repeated 3 times and further degassed by argon. Then, SphosPd-G3 (0.38 g, 0.488 mmol, 0.1 eq.) was added and capped with a septum. The mixture was further degassed under argon and was agitated for overnight at room temperature. The progress of reaction was monitored by LC/MS to complete the reaction. Aqueous NH₄Cl solution (30 mL) was added to quench the reaction. The crude was filtered by a pad of Celite to afford a clean solution. The aqueous layer was extracted with ethyl acetate (120 mL×2). The ethyl acetate layer was washed with water, brine, and dried over Na₂SO₄. After concentration, it gave crude dark yellow solid, which was purified by 5% MeOH in DCM using 120 g Gold column ISCO system to afford 3.37 g (89%) of ABZ-947. MS (ESI, pos.): Calc'd for C₃₈H₄₉N₃O₉, 691.35; found 692.3 (M+H), 1383.8 (2M+H).

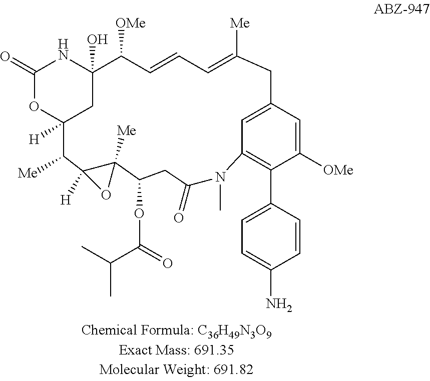 [SMILES: COc1cc2cc(c1-c1ccc(N)cc1)N(C)C(=O)C[C@H](OC(=O)C(C)C)[C@@]1(C)O[C@H]1[C@H](C)[C@@H]1C[C@@](O)(NC(=O)O1)[C@H](OC)/C=C/C=C(\C)C2]

### Synthesis of Compound B and ABZ-981:

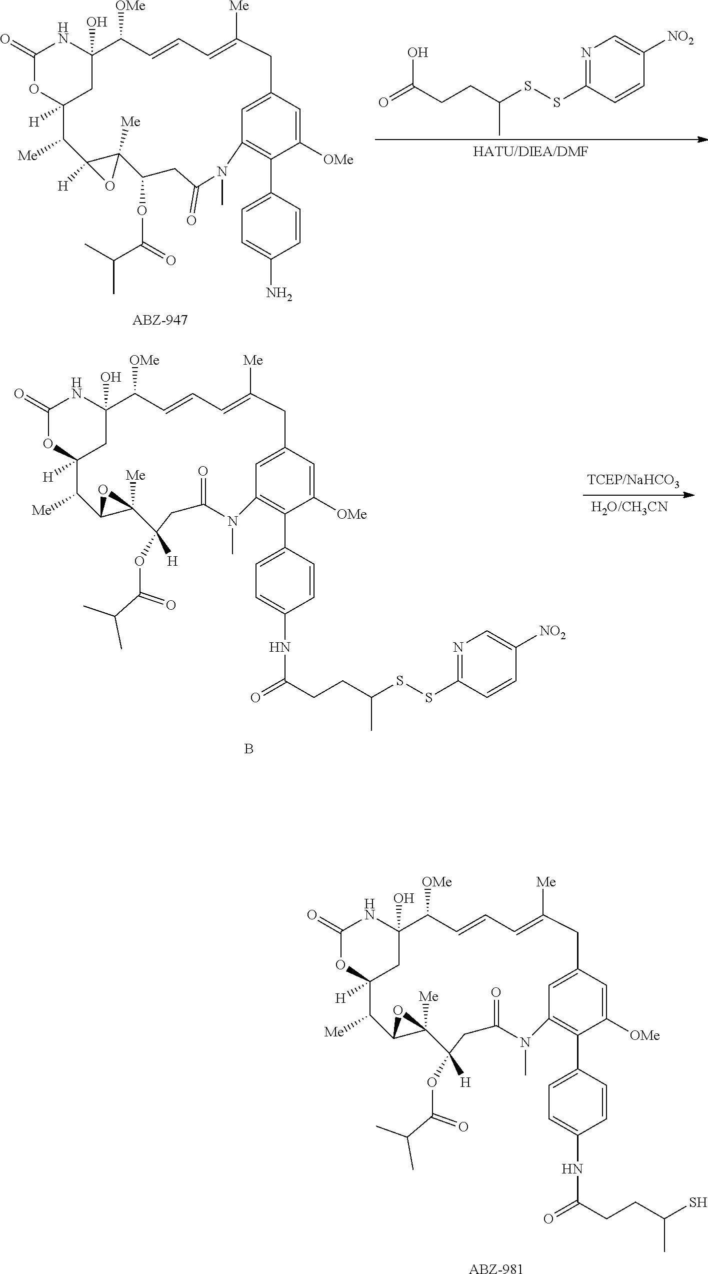

### Compound B:

To a stirred solution of ABZ-947 (237 mg, 0.0342 mmol), 4-((5-nitropyridin-2-yl)disulfanyl)pentanoic acid (101 mg, 0.753 mmol), and HATU (390 mg, 1.03 mmol) in 8 mL of anhydrous DMF was cooled to 0° C., followed by slow addition of DIEA (0.24 mL, 1.37 mmol). The resulting solution was allowed to warm to room temperature and stirred overnight. By in process of an aliquot using UPLC, it showed the complete of reaction. The reaction mixture was diluted with water (20 mL) and brine (20 mL). The mixture was extracted with ethyl acetate (50 mL×2). The organic layer was concentrated in vacuo to give crude product. The crude was diluted with DMSO (5 mL), then purified by C18 aq. 150 g ISCO column (5% ACN to 95% ACN in water, 0.05% AcOH as modifier). The pure fractions were collected, frozen, and lyophilized to yield 253 mg (77%) of Comp B as pale brown solid. MS (ESI, pos.): Calc'd for C₄₈H₅₉N₅O₁₂S₂, 961.36; found 962.42 (M+H).

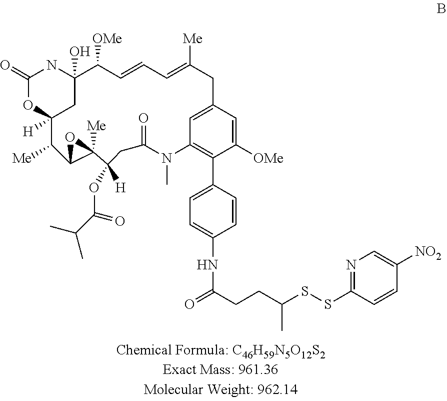 [SMILES: COc1cc2cc(c1-c1ccc(NC(=O)CCC(C)SSc3ccc([N+](=O)[O-])cn3)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

### ABZ-981:

To a solution of compound B (22 mg, 0.0207 mmol) and TCEP·HCl (59 mg, 0.207 mmol) in a mixture of ACN (1 mL) and water (0.8 mL) was slowly added saturated NaHCO₃ solution (1.2 mL) until pH reached 7-8. The resulting solution was stirred for 2 h at room temperature. LC/MS results by taking an aliquot gave the complete the reaction. The reaction mixture was concentrated under reduced pressure. The residue was diluted with 50 mL of ethyl acetate followed by addition of brine (20 mL). The organic was separated and concentrated in vacuo, which was purified by C18 aq. 50 g ISCO column (5% ACN to 95% ACN in water, 0.05% AcOH as modifier). Pure fractions were collected, frozen, and lyophilized to afford 14 mg (83%) of ABZ-981 as white solid. MS (ESI, pos.): Calc'd for C₄₃H₅₇N₃O₁₀S, 807.38; found 808.92 (M+H).

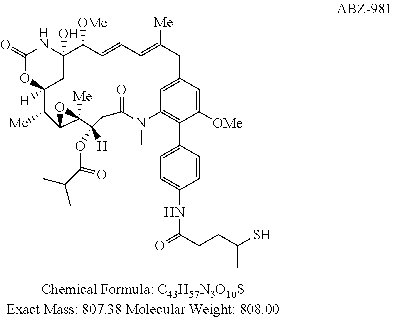 [SMILES: COc1cc2cc(c1-c1ccc(NC(=O)CCC(C)S)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

### Synthesis of Compound C and ABZ-982:

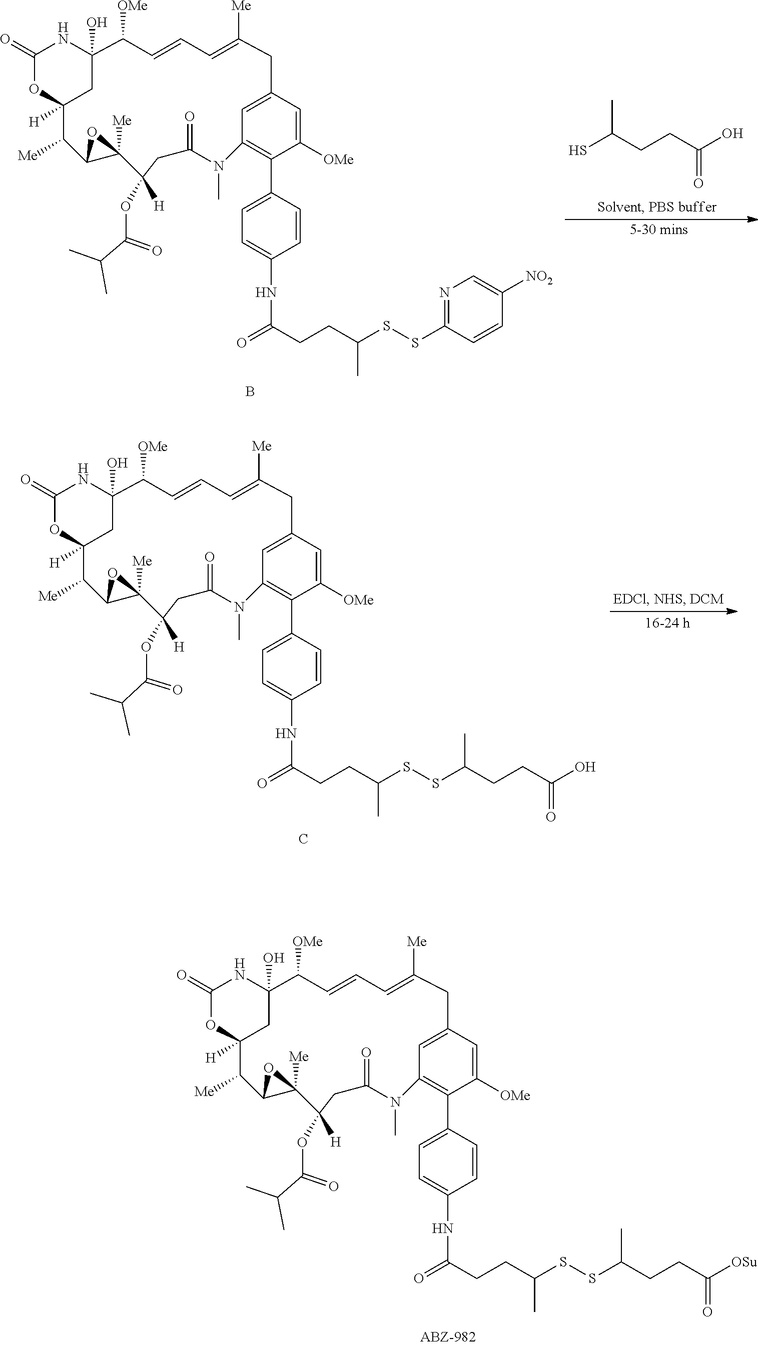

### Compound C:

To a 40 mL clear vial with compound B (420 mg, 0.436 mmol) added DMF (6 mL) and stirred to get a clear solution. Added PBS pH=7.4 buffer (2.0 mL) followed by 4-mercaptopentanoic acid (234 mg, 1.746 mmol) at rt. Stirred for 30 mins and monitored reaction progress on LC-MS (if the reaction is not completed, stir it until completion). After completion of the reaction, the reaction mixture was concentrated in vacuo, diluted with 1 mL of DMSO, and transferred to pre-equilibrated C18 aq. 100 g column. Purification was performed using eluents (10% ACN to 95% ACN in water, 0.05% AcOH as modifier). Pure fractions were collected, frozen, and lyophilized to afford 317 mg (77%) of Comp C as white solid. MS (ESI, pos.): Calc'd for C₄₈H₆₅N₃O₁₂S₂, 939.40; found 940.54 (M+H).

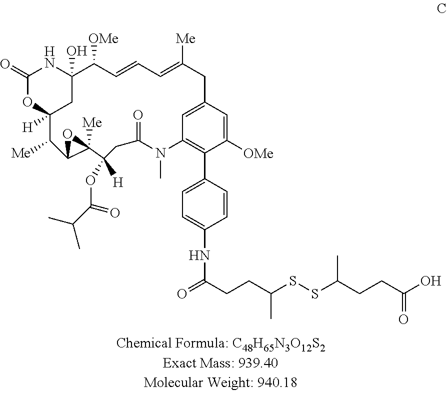 [SMILES: COc1cc2cc(c1-c1ccc(NC(=O)CCC(C)SSC(C)CCC(=O)O)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

### ABZ-982:

To a 40 mL clear vial with compound C (300 mg, 0.319 mmol) added DCM (10 mL) and stirred to get a clear solution. Added EDCI (110 mg, 0.574 mmol) followed by NHS (55 mg, 0.479 mmol) at ice-bath under argon. The resulting solution was allowed to stir for 16-24 h and monitored reaction progress by LC-MS. After completion of the reaction, the crude was concentrated under reduced pressure. The crude was dissolved with 1 mL of DMSO and plugged into pre-equilibrated C18 aq. 150 g column. Purification was performed using eluents (10% ACN to 95% ACN in water, 0.05% AcOH as modifier). Pure fractions were collected, frozen, and lyophilized to afford 280 mg (85%) of ABZ-982 as white solid. MS (ESI, pos.): Calc'd for C₅₂H₆₈N₄O₁₄S₂, 1036.42; found 1037.71 (M+H).

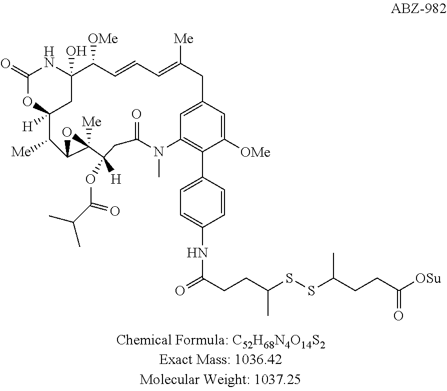

### Synthesis of Compound D and ABZ-1827:

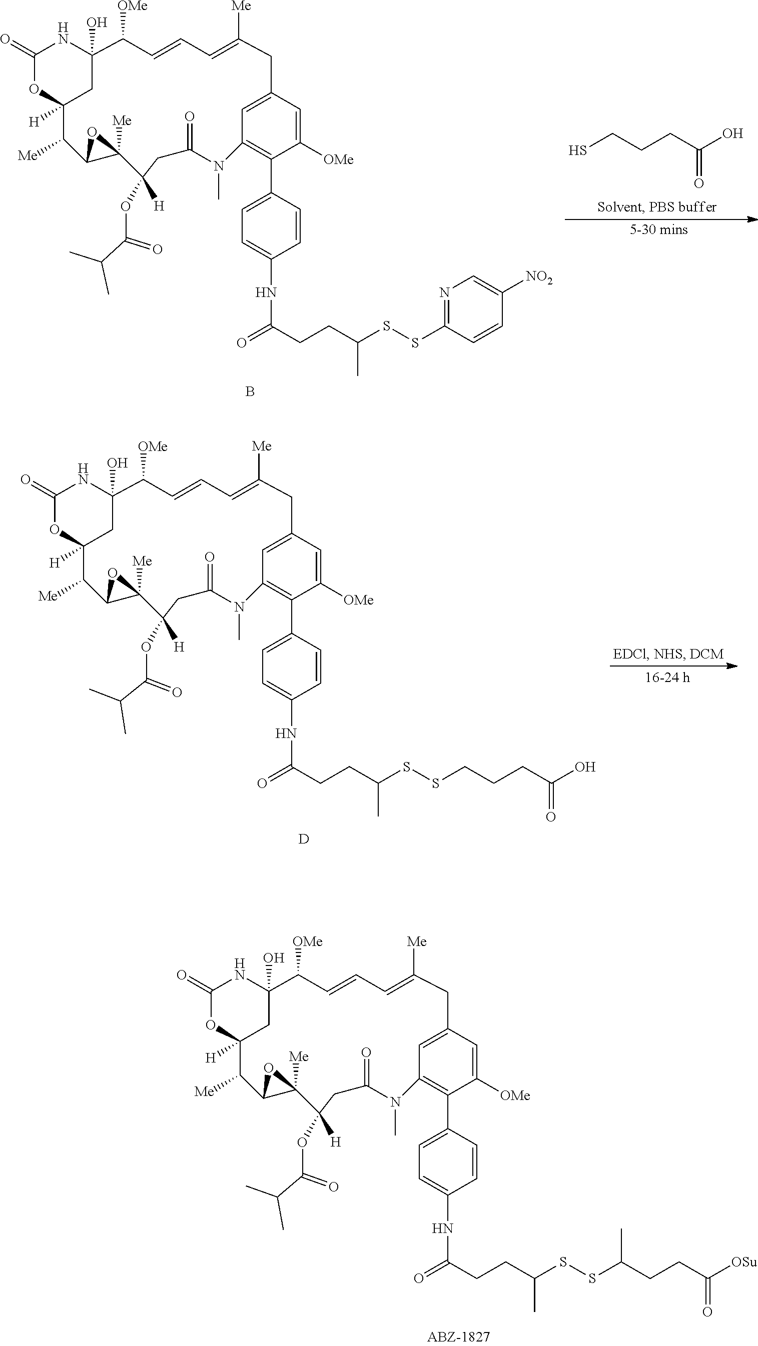

### Compound D:

To a 40 mL clear vial with compound B (500 mg, 0.519 mmol) added DMF (7 mL) and stirred to get a clear solution. Added PBS pH=7.4 buffer (2.5 mL) followed by 4-mercaptobutanoic acid (250 mg, 2.08 mmol) at rt. The dark reaction mixture was stirred for 30 mins and monitored reaction progress on LC-MS (if the reaction is not completed, stir it until completion). After completion of the reaction, the reaction mixture was concentrated in vacuo, diluted with 1 mL of DMSO, and transferred to pre-equilibrated C18 aq. 150 g column. Purification was performed using eluents (10% ACN to 95% ACN in water, 0.05% AcOH as modifier). Pure fractions were collected, frozen, and lyophilized to afford 351 mg (73%) of Comp D as white solid. MS (ESI, pos.): Calc'd for C₄₇H₆₃N₃O₁₂S₂, 925.39; found 926.73 (M+H).

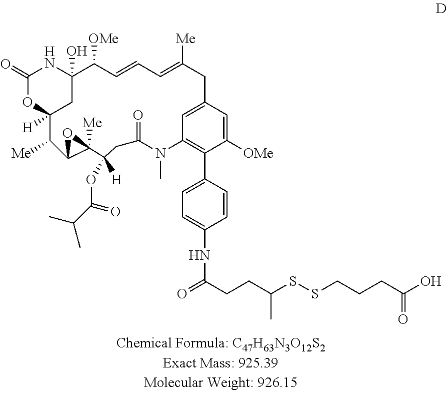 [SMILES: COc1cc2cc(c1-c1ccc(NC(=O)CCC(C)SSCCCC(=O)O)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

### ABZ-1827:

To a 40 mL clear vial with compound D (350 mg, 0.377 mmol) added DCM (10 mL) and stirred to get a clear solution. Added EDCI (130 mg, 0.680 mmol) followed by NHS (65 mg, 0.565 mmol) at ice-bath under argon. The resulting solution was allowed to stir for 16-24 h and monitored reaction progress by LC-MS. After completion of the reaction, the crude was concentrated under reduced pressure. The crude was dissolved with 1.5 mL of DMSO and plugged into pre-equilibrated C18 aq. 150 g column. Purification was completed using eluents (10% ACN to 95% ACN in water, 0.05% AcOH as modifier). Pure fractions were collected, frozen, and lyophilized to afford 270 mg (70%) of ABZ-1827 as white solid. MS (ESI, pos.): Calc'd for C₅₁H₆₆N₄O₁₄S₂, 1022.40; found 1023.75 (M+H).

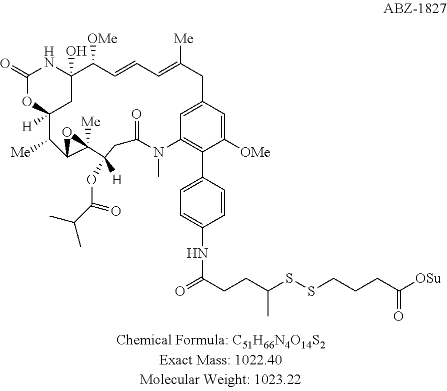

To prepare the AFP bioconjugates with different DPR values, the AFP protein was buffer exchanged into 10 mM HEPES pH 7.5 buffer with 30 kDa MWCO filter. After buffer exchange, the protein concentration was adjusted to 5 mg/mL. For the conjugation reaction, a small amount of DMSO was added to the reaction, followed by adding the ABZ982 or ABZ1827 linker-payload (freshly prepared as 10 mM stock solution with DMSO). The total amount of DMSO present in the reaction mixture was 10% (v/v), (7 eq. of linker-payloads for DPR 3-4 bioconjugate, 10 eq. of linker-payloads for DPR 5-6 bioconjugate). The reaction mixture was mixed on the tube revolver at 10 rpm/min at room temperature (22° C.) for 16-20 hours. The bioconjugates were purified by buffer exchanging the crude bioconjugate into 10 mM HEPES, 5% sucrose, pH 7.5 using 30 kDa MWCO filter. The purified bioconjugates were sterile filtered and stored at −80° C.

For a product with defined DPR, each conjugation reaction solution was mixed with equal volumes of 50 mM sodium phosphate, 2 M NaCl, pH 7. Each bioconjugate was eluted from the column with a gradient of 50 mM sodium phosphate, pH 7, 20% isopropanol. Crude solutions were mixed with equal volumes of 50 mM sodium phosphate, 4M NaCl, pH 7 and the resulting solutions were loaded onto a ToyoPearl Phenyl-650S HIC column equilibrated with 50 mM sodium phosphate, 2M NaCl, pH 7. Fractions containing different values of DPR were pooled and concentrated. The concentrated sample was buffer exchanged into PBS, pH 7.1-7.5 and sterile filtered (0.22 um PVDF membranes). DPR assignments were based on A248/A280 absorption ratios. Average DPR was calculated from the relative peak areas of individual DPR species following HIC analysis at 280 nm.

### Bioconjugate Testing

Prior to testing lead compounds in vitro, the expression of the AFP receptor and ability of cells to bind and take up fluorescently-labelled rhAFP was established in the U937 cell line as well as other cell lines, as in the study summarized in Table 1 below. In this study, rhAFP was labeled using the procedures described in the product protocol from commercially available Alexa Fluor-647 (AF-647) protein labeling kits. Binding experiments were performed in 96-well plates and fluorescence was measured using a plate reader. Tumor cells were cultured in suspension or plated in cell culture media. Each tumor cell line was incubated with AF-647 labeled rhAFP at 37° C. for 0.5, 2 and 5 hours (n=3 replicates). Incubation was stopped by chilling on ice. For cells in suspension, cells and culture medium were collected separately by centrifugation and subjected to repeat wash steps. For plated cells, trypsin/EDTA was used for cell detachment. Most of the cell lines, with the exception of MOLT-4, showed high binding, with MCF7 (breast cancer) showing the highest binding of all cell lines tested, followed by U937 (lymphoma) and COLO-205 (colorectal cancer).

In other AFP receptor binding studies, rhAFP has been labeled with Alexa Fluor-488 or with FITC, using the FluoroTag™ FITC Conjugation Kit, and achieved similar results

TABLE 1
Binding of ACT-101 to Human Tumor Cell Lines

| | | Highest AFP binding |
|---|---|---|
| | Tumor | over 5 h Period |
| Human Cancers | Cell lines | (ng/million cells) |
| Ovary adenocarinoma | OVCAR-3 | 71.0 |
| | SKOV-3 | 31.3 |
| Non-Small cell lung | H522 | 71.1 |
| adenocarcinoma | | |
| Metastatic colorectal | COLO 205 | 75.0 |
| adenocarcinoma | | |
| Breast adenocarcinoma | MCF7 | 126 |
| Acute lymphoblastic leukemia | MOLT-4 | 19.0 |
| Histiocytic lymphoma | U-937 | 84.3 |

A series of bioconjugates containing DM1, DM3, DM4 or ABZ981 synthesized under varying reaction conditions, resulting in different average DPRs. The cytotoxicity of these bioconjugates were evaluated in vitro in a panel of tumor cell lines (leukemia, breast, colorectal, ovarian and lung). Results are summarized in Table 2 below.

Of the Series 1 bioconjugates, the ABZ981 conjugate had the highest DPR (7.5) but had a similar potency in the U937 cell line as the other conjugates synthesized at a lower DPR (4.4-5.1). The DM4 conjugate (DPR 4.4) also had a similar potency in the MCF-7 cell line as the ABZ981 conjugate. However, the potency DM1 and DM3 conjugates was approximately 3-fold lower in this cell line.

In the Series 2 conjugates which were synthesized at a similar DPR (3.5-4), the DM4 conjugate was the most potent across the cell lines examined while the ABZ981 conjugate (DPR 3.5) was 3 to 15-fold less potent.

These results indicated that the potency in vitro is not necessarily related to the DPR.

TABLE 2
Potency of bioconjugates of varying DPR in various human tumor cell lines

| ToxIn | SerIes 1 | | Average IC50 [nM] | | SerIes 2 | | Average IC50 [nM] | | | |
|---|---|---|---|---|---|---|---|---|---|---|
| released | Lab codes | DPR | U937 | MCF-7 | Lab codes | DPR | COLO-205 | SKOV-3 | NCl-H522 | OV AR3 |
| DM4 | HM023-8 | 4.4 | 1.33 | 1.37 | AH00-1D | 3.9 | 4.1 | 4.83 | 7.26 | 2.99 |
| DM1 | HM023-6 | 5.1 | 1.6 | 4.62 | AH00-2D | 3.9 | 8.93 | 21.25 | 18.41 | 4.88 |
| DME | HM023-2 | 4.8 | 1.6 | 4.62 | AH00-3D | 4 | 10.74 | 22.8 | 21.88 | 5.47 |
| ABZ981 | HM026-2 | 7.5 | 1.42 | 1.32 | AH001 11D-c | 3.5 | 21.02 | 74.13 | 52.74 | 9.74 |

An ex vivo study performed incubating the Series 2 AFP bioconjugates incubated in mouse and human serum at 37° C. for 3 and 7 days respectively. Bioconjugates were captured on Innova magnetic beads charged with an anti-AFP antibody. Captured samples were analyzed by LC-ESI-MS to confirm DPR profiles and integrity of the protein. All bioconjugates showed good stability with an average reduction in DPR of −5.9% to −13.5% in mouse serum after 3 days and an average reduction in DPR of −8.1% to 15.8% after 7 days in human serum.

### Biodistribution/Pharmacokinetics in Tumor-Bearing Mice

Bioconjugates containing DM4, DM1 or ABZ981 were resynthesized at a DPR of approximately 4 (Table 3) for comparative testing in a pharmacokinetic/biodistribution (PK/BD) study in mice subcutaneously implanted with COLO-205 tumor xenografts.

TABLE 3
Description of bioconjugates tested in the PK/BD study

| | | Concentration | Endotoxin | Monomeric | Average |
|---|---|---|---|---|---|
| | | of Test Article | Content | Species | Toxin-Protein |
| Toxin released | Lab code | (mg/mL) | (EU/mg) | (%) | Ratio (DPR) |
| DM4 | ALP100-AH004-1 | 4.10 | <0.12 | 97.2 | 4.1 |
| DM1 | ALP100-AH004-2 | 4.83 | <0.10 | 98.4 | 4.0 |
| ABZ981 | ALP100-AH004-11 | 4.20 | 0.13 | 97.4 | 4.0 |

The volumes of administration were adjusted to deliver the same amount of bioconjugate (25 mg/kg) to each group of animals as a single IV injection into the tail vein. Blood samples were taken at 0.25 and 1 hours post-injection and blood and tissue samples were taken at 4, 8 and 24 hours to evaluate the pharmacokinetics and biodistribution of the bioconjugates (n=3 per timepoint). A commercially available ELISA kit was used to measure AFP levels and levels of free maytansine and its metabolites were measured by LC/MS. Negligible amounts of free toxin in blood were detected over this time period (≤0.001% of the total dose injected), consistent with the ex vivo stability of the bioconjugates in mouse serum.

### Tumor Uptake of Bioconjugates and Toxin Release and Metabolism

Delivery of toxin to the tumor (free maytansine plus metabolites) was highest with the AFP-ABZ981 conjugate relative to DM-based conjugates, at all time points compared to other groups when expressed either as ng/mg tissue or as a percent of total dose administered. Levels achieved with the AFP-ABZ981 conjugate were approximately 3 and 22 times higher than the DM1 and DM4 conjugates respectively. This was unexpected, as the AFP-ABZ981 conjugate was not more potent in vitro compared to the DM4 conjugate. Peak free toxin levels in the AFP-ABZ981 conjugate group when normalized to AFP were 18% of the AFP levels in tumor, suggesting that a significant percentage of the toxin remains bound to AFP. However, toxin release in tumors was higher for the AFP-ABZ981 conjugate than for the AFP-DM4 and AFP-DM1 conjugates (approximately 1 and 9% respectively). The higher levels of toxin achieved with the AFP-ABZ981 conjugate may be due to less steric hindrance since there is only a single methyl group on either side of the disulfide bond with this conjugate.

Bioconjugates containing ABZ981 with two different release functionalities (AFP-ABZ982 and AFP-ABZ1827) were subsequently prepared at DPRs of 2.5 to 6.3. The results of in vitro testing in the U937 and SKOV3 cell lines are shown in Table 4. Cells were incubated for 4 days in the presence of the compounds over an 8-point concentration range, with each concentration tested in triplicate. Two experiments performed using freshly thawed U937 cells gave similar results to U937 cells maintained in culture.

TABLE 4
Cytotoxicity of AFP-ABZ982 and AFP-ABZ1827 bioconjugates

| | | Average IC₅₀ (nM) | | |
|---|---|---|---|---|
| | | U937 | U937 freshly | SKOV3 |
| | | cultured | thawed cells | cells |
| Conjugate | Average DPR | cells (n = 1) | (n = 2) | (n = 1) |
| AFP-ABZ982 | 2.50 | 5.22 | 5.25 | 56.13 |
| AFP-ABZ982 | 3.84 | 4.49 | 5.29 | 35.54 |
| AFP-ABZ982 | 5.51 | 2.01 | 1.71 | 17.47 |
| AFP-ABZ982 | 6.28 | 6.55 | — | 37.03 |
| AFP-ABZ1827 | 3.35 | 5.52 | 7.07 | 66.38 |
| AFP-ABZ1827 | 4.40 | 7.47 | 5.98 | 58.84 |
| AFP-ABZ1827 | 5.88 | 2.04 | 2.93 | 35.65 |
| Free Toxin | | 3.06 | 2.84 | 5.53 |
| (ABZ- 981) | | | | |

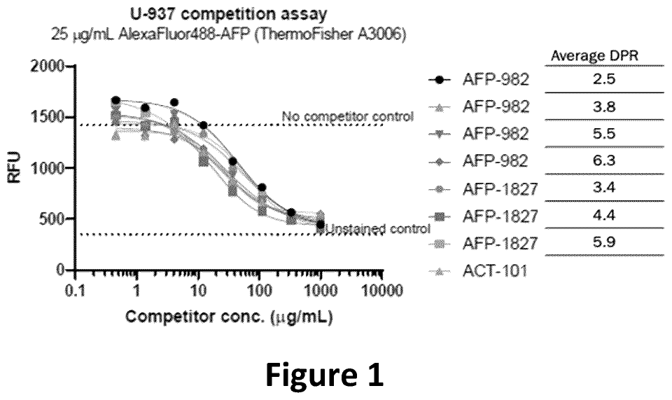

The specific binding of these bioconjugates to the AFP receptor was confirmed by competition experiments in the U937 cell line using fluorescently-labelled rhAFP (FIG. 1). In brief, U-937 cells in cultured were transferred to serum free medium for 2 hours at 37° C., co-incubated with AlexaFluor488-labeled rhAFP and a titration of unlabeled rhAFP or bioconjugates for 1 hour at 4° C. Cells were washed, fixed and analysed by flow cytometry.

Turning to Table 5, two bioconjugates of each of AFP-ABZ982 and AFP-ABZ1827 (4 in total) were prepared at DPRs of approximately 4 (“low DPR”) and 6 (“high DPR”) for efficacy testing in vivo in the COLO-205 human tumor xenograft model. Potency in the COLO-205 cell line prior to implantation was assessed. Briefly, 96 well plates were seeded with COLO-205 cells either at 5,000 or 2,500 cells per well in a total volume to 50 μL per well. Plates were incubated overnight then treated with the bioconjugate at 37° C. for 72 hours and then a CellTiter-Glo assay was performed to measure cell viability. Samples were run in triplicate. The high DPR bioconjugates were more potent than the low DPR bioconjugates and showed similar potency against COLO-205 in vitro, although the AFP-ABZ982 high DPR bioconjugate was the most potent, with a lower IC50 than even the unconjugated toxin-linker.

TABLE 5
Potency of bioconjugates against COLO-205 cells in vitro

| | | Average | Monomeric | Average |
|---|---|---|---|---|
| Conjugate | Lab Code | DPR | species (%) | IC₅₀ (nM) |
| AFP-ABZ982 | ABZ-2626 | 3.74 | 94 | 12.9 |
| low DPR | | | | |
| AFP-ABZ982 | ABZ-2627 | 5.81 | 95 | 4.1 |
| high DPR | | | | |
| AFP-ABZ1827 | ABZ-2624 | 3.92 | 98 | 17.2 |
| low DPR | | | | |
| AFP-ABZ1827 | ABZ-2625 | 5.96 | 94 | 5.9 |
| high DPR | | | | |
| ABZ-981 | SC-201-027-1 | — | | 6.1 |
| toxin-linker | | | | |

### Efficacy in Tumor-Bearing Mice

In the present experiments, the efficacy of four novel AFP-maytansinoid bioconjugates of differing drug-protein ratios (AFP-ABZ982 high DPR/low DPR and AFP-1827 high DPR/low DPR) and slightly different linker structures. The four ABZ981-containing bioconjugates above were resynthesized and formulated in a 10 mM HEPES buffer, 5% sucrose at pH 7.5 (Table 6). Prior to testing the efficacy in tumor-bearing mice, the maximum tolerated dose (MTD) was determined for each bioconjugate in healthy mice, employing the same dosing regimen used for the efficacy study (Q1D×5 for 2 weeks) at doses of 0, 5, 10, 20 and 40 mg/kg. MTD was based on clinical observations made daily during the dosing period and elevations of liver enzymes (AST/ALT) determined at the end of treatment. Surprisingly, the MTD of AFP-ABZ1827 high DPR was higher than the MTD for the low DPR. In general, a bioconjugate with a higher DPR which carries a larger amount of toxin would be expected to have a lower MTD.

TABLE 6
Description of Bioconjugates tested in MTD and Efficacy Studies

| | | | | Endotoxin | Monomeric |
|---|---|---|---|---|---|
| | | Dose | | Content | Species |
| Group No. | Conjugate | (MTD) | DPR | (EU/mg) | (%) |
| Group 1 | AFP-ABZ982 | 20 mg/kg | 3.74 | <0.2 | 94 |
| | low DPR | | | | |
| Group 2 | AFP-ABZ982 | 10 mg/kg | 5.81 | <0.2 | 95 |
| | high DPR | | | | |
| Group 3 | AFP-ABZ1827 | 10 mg/kg | 3.92 | <0.2 | 98 |
| | low DPR | | | | |
| Group 4 | AFP-ABZ1827 | 20 mg/kg | 5.96 | <0.2 | 94 |
| | high DPR | | | | |

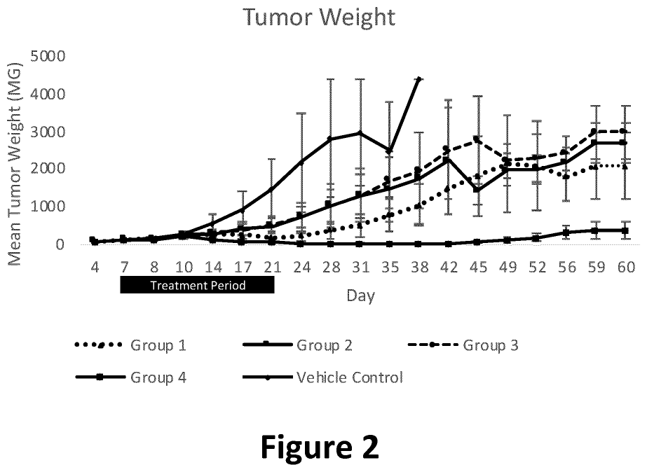

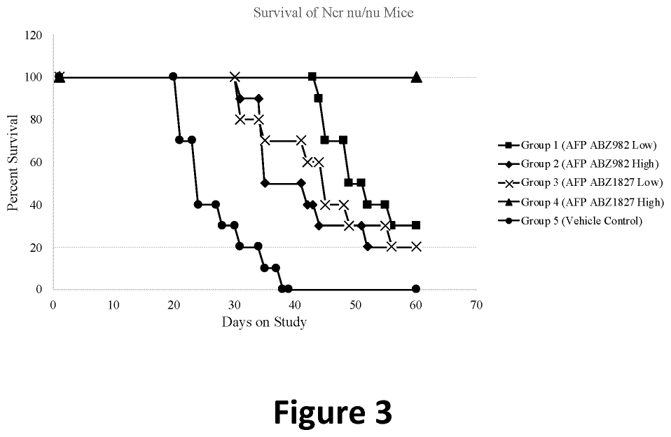

In the colon carcinoma xenograft model, 7 week old athymic male mice were implanted with 1×10⁷ COLO-205 cells subcutaneously in the right flank. Mice with tumors of approximately 100 mm³ to 200 mm³ were randomized to receive control (vehicle) or one of the 4 bioconjugates (10 animals/group). Animals were treated daily for 2 weeks with 2 days of rest after 5 doses; tumor volume was assessed twice weekly for 60 days following implantation. FIGS. 2 and 3 reveal the valuable properties of the present bioconjugates in the COLO 205 human xenograft model of colon cancer as shown by this study.

As shown in FIG. 2, AFP bioconjugates AFP-ABZ982 and especially AFP-ABZ1827 have a dramatic, inhibitory effect on the growth of the COLO-205 tumors, especially when AFP-ABZ982 is used at low DPR and when AFP-ABZ1827 is used at high DPR. A statistically significant (p<0.05) reduction in tumor weight was observed in all treatment groups compared to control beginning at Day 17 and lasting until all control animals were euthanized. In the high DPR AFP-ABZ1827 group, tumor regression occurred earlier (Day 14) and continued following treatment discontinuation with tumor volumes falling below the limit of detection in 9 of 10 animals.

As shown in FIG. 3 all bioconjugates tested improved overall survival of tumor-bearing mice when compared to vehicle control. In the AFP-ABZ1827 high DPR group, all mice were alive at the end of the 60 day observation period whereas all animals in the control group were dead by day 38 due to uncontrolled tumor growth. No signs of toxicity were observed in treated mice.

In this study the high DPR AFP-ABZ1827 was superior to the low DPR equivalent and all other bioconjugates tested, in the context of reducing tumor weight and improving overall survival of tumor bearing mice.

Further studies of high DPR AFP-ABZ1827 (ACT-903) at different dose levels showed in the COLO-205 xenograft model showed significant reduction in tumor growth by Day 14 after a single IV injection at a dose of either 30 mg/kg, 40 mg/kg or 50 mg/kg (p<0.05). In this study a second dose was administered on Day 24 for these two dose groups. This regimen was associated with a significantly prolonged survival in the 40 mg/kg group (p<0.001) and 50 mg/kg group (p=0.0037) compared to the vehicle control group.

Further studies of high DPR AFP-ABZ1827 (ACT-903) were carried out in ovarian cancer organoids derived from two patients. Studies using fluorescently labeled ACT-101 confirmed the presence of the AFP receptor as the compound bound to and was rapidly taken up by ovarian organoids. ACT-903 effectively induced cell death in a concentration- and time-dependent fashion in both organoids. Using AnnexinV staining, massive apoptosis was observed by 72 hr even at low concentrations of ACT-903, similar to the positive controls used in the experiment, known cytotoxins thapsigargin and staurosporine, whereas the unconjugated protein (ACT-101) was innocuous to the cells.

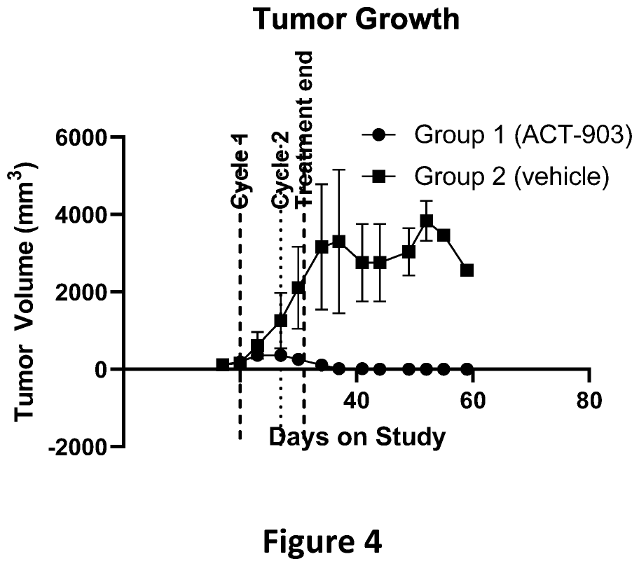

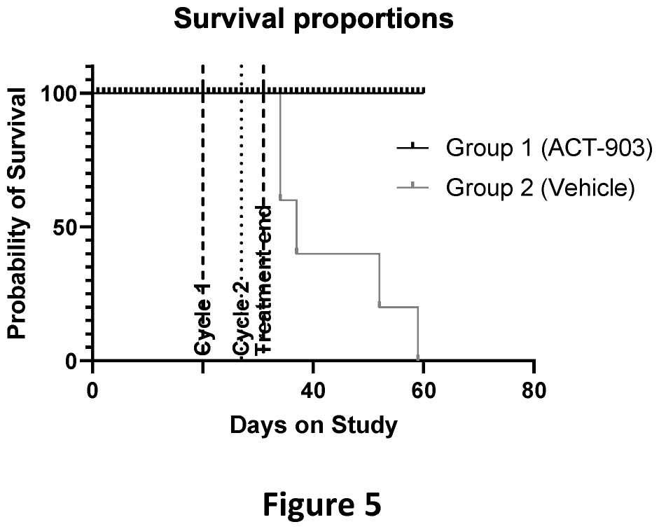

Further studies of high ACT-903 were carried out in the ovarian carcinoma xenograft model. Five to 7 week old athymic female mice were implanted with A2780 ovarian tumor cells subcutaneously in the right flank. Mice were randomized to receive control (vehicle) or ACT-903 (5 animals/group). Animals were treated daily for 10 days with 2 days of rest after 5 doses. Tumor volume at baseline prior to treatment ranged from 108 mm³ to 288 mm³. Tumor volume was assessed twice weekly for 60 days following implantation. FIGS. 4 and 5 reveal the valuable properties of the present bioconjugates in the A2780 human xenograft model of ovarian cancer as shown by this study.

As shown in FIG. 4, ACT-903 has a dramatic, inhibitory effect on the growth of the A2780 tumors. The tumor growth curves indicate a significant (p<0.0001) reduction in tumor burden in ACT-903 treated group, with complete tumor regression (tumors not visible or palpable) occurring in all mice following treatment. Furthermore tumor regrowth did not occur after treatment ended during the 60 day observation period.

As shown in FIG. 5, ACT-903 significantly improves survival compared to vehicle control (p<0.0001), with all mice in the ACT-903 treated group surviving to the end of the 60-day observation period, compared to no surviving mice in the control group.

It will thus be appreciated that the AFP receptor is a highly attractive novel target for cancer therapeutics since its expression is generally very low or absent in normal tissues, except MDSCs, but is present in large numbers on many common cancers. Furthermore, since AFP is a naturally occurring human protein to which every human fetus is exposed in utero, there is reduced risk of any harmful immune reaction to a rhAFP-cytotoxin bioconjugate.

The bioconjugates described herein contain a disulfide linker which can be reduced by glutathione, which is present in high concentrations inside tumor cells, but at low concentrations in the blood stream. Thus, the present bioconjugates will offer a dual tumor-targeting mechanism based on both differential expression of the AFP receptor on tumors and increased glutathione concentration in tumors.

Conjugates including those claimed herein can be taken forward into additional animal models, including AFP-ABZ982 low DPR and high DPR, as well as the preferred AFP-ABZ1827 low DPR and especially high DPR. These represent different release functionalities and DPR loading. Potency in vitro is in the low nM range—as high as the free cytotoxin in the U937 cell line. Dimer formation is not an issue under the conjugation conditions used, and bioconjugates are stable for at least 96 hours at 20° C. when formulated in either 10 mM HEPES or Tris-HCl buffer with 5% sucrose and show high serum stability at 37° C. over 7 days in mouse/human serum.

All references, including publications, patent applications, and patents that are cited herein are hereby incorporated by reference.

1-101. (canceled)

102. A bioconjugate useful in the treatment of diseased cells, comprising
(1) an agent that binds to alpha-fetoprotein (AFP) receptor (AFPR) on the surface of a cell;
(2) a cytotoxin (CT) that is toxic to the cell, and
(3) a glutathione-cleavable disulphide linker, wherein the linker
(i) is coupled covalently between the AFPR-binding agent and the CT, and
(ii) releases the CT intracellularly to the cell.

103. The bioconjugate according to claim 102, wherein the AFPR-binding agent has the amino acid sequence of mature, wildtype AFP, of an AFPR-binding fragment or of AFPR-binding variant of the mature wildtype AFP or of the AFP fragment.

104. The bioconjugate according to claim 103, wherein the AFP is recombinant human AFP, or an AFPR-binding fragment or variant thereof, the variant comprising from 1-5 amino acid substitutions.

105. The bioconjugate according to claim 102, wherein the agent is recombinant [Asn²³³Gln] mature human AFP comprising SEQ ID No.3.

106. The bioconjugate according to claim 102, wherein the agent comprises SEQ ID No.4.

107. The bioconjugate according to claim 105 wherein the cytotoxin (CT) has the formula:

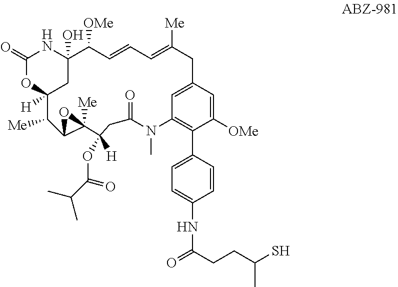 [SMILES: COc1cc2cc(c1-c1ccc(NC(=O)CCC(C)S)cc1)N(C)C(=O)C[C@H](OC(=O)C(C)C)[C@]1(C)O[C@H]1[C@H](C)[C@@H]1C[C@@](O)(NC(=O)O1)[C@H](OC)/C=C/C=C(\C)C2]

108. The bioconjugate according to claim 105, wherein the linker coupled between the AFPR-binding agent and the cytotoxin is selected from:
—S—CH(CH3)-(CH2)₂-CO—; and Linker 1
—S—(CH2)₃-CO—. Linker 2

109. A conjugate useful in the production of a bioconjugate according to claim 102, wherein the conjugate comprises the linker-cytotoxin shown below:

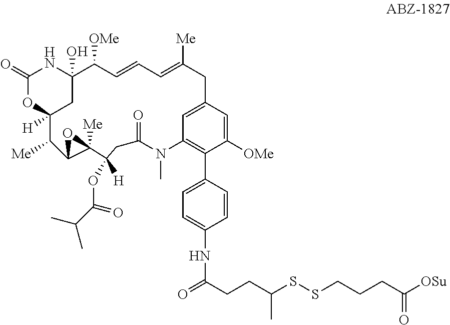

110. A bioconjugate of the formula: 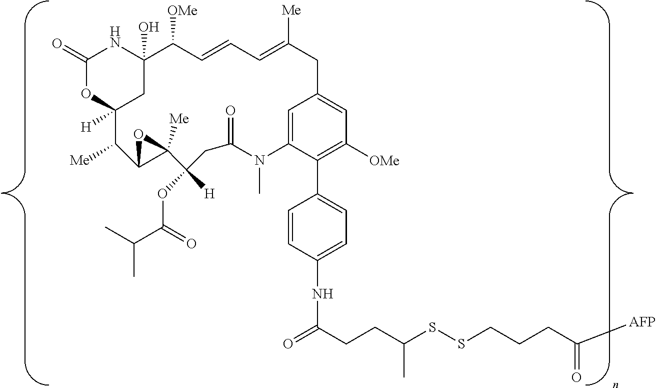
wherein AFP is an AFP receptor-binding form of alpha-fetoprotein.

111. A preparation according to claim 110 where the average DPR is between 4 to 8.

112. A preparation according to claim 110 where the average DPR is about 5.8.

113. A pharmaceutical composition comprising a pharmaceutically acceptable carrier and a bioconjugate according to claim 110.

114. The pharmaceutical composition according to claim 113, wherein the pharmaceutically acceptable carrier is 10 mM HEPES buffer at pH 7.5 with 5% sucrose.

115. The pharmaceutical composition according to claim 114, for administration into a subject suffering from a cancer that is AFPR+.

116. A method for treating AFPR+ cells in a subject in need thereof, the method comprising administering to the subject a treatment effective amount of a pharmaceutical composition according to claim 111, wherein the AFPR+ cells are cancer cells.

117. A method for treating AFPR+ myeloid-derived suppressor cells in a subject in need thereof, the method comprising administering to the subject a treatment effective amount of a pharmaceutical composition according to claim 111, wherein the AFPR+ cells are MDSCs.

118. For use in therapeutic combination, for the treatment of subjects suffering from cancers that are AFPR+, a pharmaceutical composition as defined in claim 111 and a second treatment agent.

119. The use according to claim 118, wherein the second treatment agent is an immune checkpoint inhibitor or a CAR-T agent or another immune-oncology therapy.

120. A process for producing a bioconjugate, comprising coupling an AFPR-binding form of AFP according to claim 105 with a conjugate according to claim 109.

121. A process for producing a bioconjugate whereby the cytotoxin ABZ-981 is first coupled with a glutathione-sensitive disulfide linker to produce an intermediate: 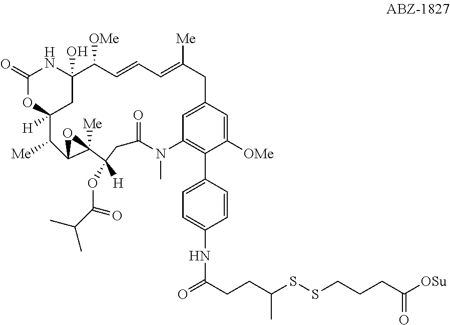
that is then reacted with an AFPR-binding form of AFP that is recombinant [Asn²³³Gln] mature human AFP comprising SEQ ID No.3, and the intermediate is coupled covalently to the epsilon amino groups of lysine residues in the AFP.


SEQUENCE LISTING (4 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 3 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVQFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 4 - protein, 609 aa, Homo sapiens
  SITE 251: note = MISC_FEATURE - X is Q
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVXFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV